(19) United States
(12) Patent Application Publication
(10) Pub. No.: US 2005/0260618 A1
(43) Pub. Date: Nov. 24, 2005

(54) Detection of ruminant DNA via PCR

(75) Inventors: James Cullor, Woodland, CA (US); Wayne Smith, Fairfield, CA (US); Gabriel Rensen, Sacramento, CA (US); Mary Sawyer, Winters, CA (US); Bennie Osburn, Davis, CA (US); Alice Wong, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/046,190

(22) Filed: Jan. 28, 2005

Related U.S. Application Data
(60) Provisional application No. 60/540,757, filed on Jan. 30, 2004.

(51) Int. Cl. C12Q 1/68; C12P 19/34

(52) U.S. Cl. 435/6; 435/91.2

(57) ABSTRACT

The present invention provides methods, compositions and kits for amplifying, measuring, and or detecting ruminant DNA in samples.

### CROSS-REFERENCES TO RELATED APPLICATIONS

This application claims the benefit of U.S. Provisional Patent Application No. 60/540, 757, filed Jan. 30, 2004, the disclosure of which is incorporated by reference in its entirety for all purposes.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

Bovine spongiform encephalopathy (BSE) or “Mad Cow” disease was first recognized in Great Britain in 1986 and spread to countries on the European continent (see, e.g., Anderson et al., Nature 382:779-88 (1996)). Subsequent epidemiological studies have identified rendered material from scrapie infected sheep into bovine feeds as the most probable initial cause of BSE. The pathogenic agent of BSE, i.e., prions were spread to cows from the rendered offal. BSE was further propagated by the inclusion of rendered bovine meat and bone meal (BMBM) as a component of animal feeds (see, e.g., Wilesmith et al., Vet Rec. 123:112-3 (1988)). BSE has now been identified in the United Kingdom, Europe, Japan, and North America, including Canada and the United States (see, e.g., Normile, Science, 303:156-157 (2004)).

In 1997, in response to epidemiologic evidence regarding the transmission of BSE, the Food and Drug Administration of the United States (FDA) prohibited the incorporation of certain mammalian tissues (e.g., tissue derived from the CNS, and intestinal tissue) in ruminant feed (see, e.g., 62(108) Federal Register 30935-78 (Jun. 5, 1997)). Products believed to pose a minimal risk, including blood, blood products, gelatin, milk and milk products, protein deprived solely from swine or equine sources and inspected meat products offered for human consumption were initially exempted from the ban. In January of 2004, the USDA prohibited the incorporation of “specified risk materials,” i.e., skull, brain, trigeminal ganglia, eyes, vertebral column, spinal cord, and dorsal root ganglia of cattle 30 months and older, as well as tonsils and distal ileum of the small intestine from cattle of any age into any human food, including any food that is likely to enter the human food supply. In the same month, the FDA extended the ban to mammalian blood and blood products, uneaten meat and other scraps from restaurants from ruminant feed.

In addition, the FDA has advised that that bovine derived materials from animals born in or residing in countries where BSE had occurred should not be used to manufacture FDA-regulated products intended for administration to humans (including, e.g., vaccines). The FDA has also recommended that the use of high-risk cattle-derived protein be avoided in the manufacture of cosmetics

Currently, estimates of compliance are based on an honor system accompanied by signatures and FDA site visits in which manufacturing protocols and record keeping are checked. The tests for verification currently available for determining the presence of ruminant source proteins in animal feed is a time consuming microscopic examination method (Tartaglia et al., J Food Prot. 61(5):513-518 (1998)) which has a lower limit of detection greater than 5% by weight of feed or immunological assays with a reported detection limit of 1%-5% by weight (“Reveal®” Neogen Corp., Lansing Mich.).

Since the initial bans were implemented, development of methods for extracting and identifying banned additives in samples (e.g., ruminant feed, pet food, cosmetics, human food, and nutraceuticals) has been given a great deal of attention by researchers. For example, Tartaglia et al., J. Food Prot. 5:513-518 (1998); Wang et al., Mol. Cell Probes 1:1-5 (2000); and Kremar and Rencova, J. Food Prot. 1:117-119 (2001) describe methods of extraction and identification of bovine mitochondrial DNA. Myers et al., J. Food Prot. 4:564-566 (2001) compared methods of nucleic acid extraction. However, none of these methods address the issue of inhibitors present in the feeds which interfere with detection of the DNA, thus causing a high incidence of false negative results. A commercial kit is available which addresses the presence of PCR inhibitors (Qiagen Stool Kit, Qiagen Inc, Valencia Calif., 91355), but as discussed in the examples below, use of this kit does not eliminate all PCR inhibitors present in animal feeds. A commercial screening kit based on an enzyme labeled immuno-assay system (ELISA) identifies ruminant contamination in cattle feeds (Neogen AgriScreen, Lansing Mich., 48912), but this kit depends on the presence of ruminant protein in the cattle feed and does not address the issue of minute quantities of ruminant protein that may be in the feed.

The application of the polymerase chain reaction (PCR) of mitochondrial DNA (mtDNA) has been investigated for detecting the presence of bovine contamination in ruminant feed (Tartaglia et al., J Food Prot. 61(5):513-518 (1998)). However, the procedure failed to detect contamination levels below 0.125% by weight, and required an overnight incubation step. The investigators also suggested an additional step utilizing restriction endonuclease analysis of the amplified product to insure the specificity of the amplified product.

False negative results which fail to detect the presence of banned ruminant protein in the animal food supply, the human food supply, vaccines, nutraceuticals, or cosmetics, could lead to the contamination of these substances with the banned ruminant protein, either directly or indirectly. Such contamination could have a significant adverse impact on public health by increasing the risk of BSE. In addition, the higher risk of contamination has potentially devastating effects on the food, cosmetic, and vaccine industries by drastically increasing the costs associated with monitoring their products ruminant material. More sensitive tests to detect ruminant material in any food, vaccines, or cosmetics before they enter the food, vaccine, or cosmetic would both increase the efficiency of monitoring food, vaccines, or cosmetics for contamination by ruminant material and greatly reduce the risk of BSE to the general public.

Thus, there is a need in the art for additional methods and compositions for detecting ruminant DNA. In particular, there is a need for more sensitive and accurate methods for detecting ruminant DNA, which reduces and/or eliminates false negatives. The present invention addresses these and other needs.

### BRIEF SUMMARY OF THE INVENTION

The present invention provides methods and kits for amplifying, measuring and/or detecting ruminant DNA in samples.

One embodiment of the invention provides a method of amplifying ruminant DNA in a sample (e.g., of an animal feed, an animal feed component, a cosmetic, a nutraceutical, a vaccine, a colloidal infusion fluid, or combinations thereof) by contacting nucleic acid from the sample with an RNase (e.g., RNase A, RNase B, RNase D, RNase E, RNase H, RNase I, RNase P, RNase S, RNase T, RNase V, and combinations thereof) to generate RNase-treated nucleic acid; amplifying the RNAse-treated nucleic acid using a first ruminant-specific primer and a second-ruminant-specific primer to amplifying ruminant DNA present in the sample, thereby producing a first amplified ruminant DNA. In some embodiments, the methods further comprise detecting the amplified ruminant DNA. In some embodiments, the methods further comprise amplifying the first amplified ruminant DNA with a third ruminant specific primer and a fourth ruminant specific primer. In some embodiments, the nucleic acid is isolated from the sample prior to contacting said nucleic acid with an RNase. In some embodiments, the ruminant DNA being detected is from a cow, a sheep, a goat, an elk, a deer, and combinations thereof. In some embodiments, the RNase-treated nucleic acid is generated by contacting said isolated nucleic acid with said RNase at about 30° C. to about 40° C. for about 15 minutes to about 120 minutes. In other embodiments, the RNase-treated nucleic acid is generated by contacting said isolated nucleic acid with said RNase at about 37° C. for about 60 minutes. In some embodiments, the ruminant DNA comprises a mitochondrial DNA sequence (e.g., cytochrome c, cytochrome b, 12S RNA, ATPase subunit 8, ATPase subunit 6, ATP synthetase, subunit 8, and subsequences thereof). In some embodiments, the ruminant-specific primer pairs are SEQ ID NOS:1 and 2; SEQ ID NOS:3 and 4; or SEQ ID NOS:11 and 12. In some embodiments, the sample is an animal feed (e.g., bovine tallow, milk or a fraction thereof). In some embodiments, the animal feed is cattle feed (e.g., comprising about 0.5% to about 30%, about 0.75% to about 20%, or about 1% bovine tallow). In some embodiments, the methods further comprise detecting the amplified product (e.g., by detection of a signal from a fluorophore bound to the amplified product or by detection of a signal from an oligonucleotide probe bound to the amplified product).

Another embodiment of the invention also provides a kit for detecting ruminant DNA. The kits typically comprise at least one pair of ruminant-specific primers, RNase (e.g., RNase A, RNase B, RNase D, RNase E, RNase H, RNase I, RNase P, RNase S, RNase T, RNase V, and combinations thereof) and instructions for use. In some embodiments, the kits further comprising a second pair of ruminant-specific primers.

A further embodiment of the invention comprises isolated nucleic acids comprising the nucleic acid sequences set forth in SEQ ID NOS:1, 2, 3, 4, 11, 12, 13, or 14.

The compositions and methods of the present invention are described in greater detail below.

### BRIEF DESCRIPTION OF THE DRAWINGS

FIG. 1 depicts data from melting point analysis of the amplified products described in Example 4.

FIG. 2 is a table (Table 1) summarizing the inhibitory effects of contaminants on amplification of nucleic acid. Inhibition of PCR was determined using picogram amounts of control DNA (human DNA—HDNA). Minimum picogram amounts of HDNA varied one hundred fold among the seven undiluted cattle feed extracts. Diluting the extracts (1:100) increased the amplification of the detected HDNA. The minimum detection level was improved in cattle Feed Nos. 2, 3, 4, and 6 by 10 fold; Feed Nos. 1, 5, and 7 remained the same.

FIG. 3 is a table (Table 2) summarizing the analyses of the purity of the DNA extracted from cattle feed. The determinations to assess the amount and purity of the extracted material detected the presence of substances other than DNA. Boiling and centrifugation of the extracts had no effect on the amount of non-specific DNA, the 260/280 nm ratio or on the PCR result. The average 260/280 nm spectrophotometer ratio was 2.11 (STD DEV: +/−0.09; range: 1.40 to 2.37) and 4/126 extracts were below 1.8. The ratio of >2.0 implicated RNA as a possible contaminant. The disparity between the DNA (fluorometer determinations) and nucleic acid (spectrophotometer calculations) was from 10 μg/ml to 40 μg /ml times greater in the nucleic acid content. Gel electrophoresis demonstrated that treatment of the extracts with RNAse removed RNA while DNA bands and a band of molecular weight below 2,000 bp remained.

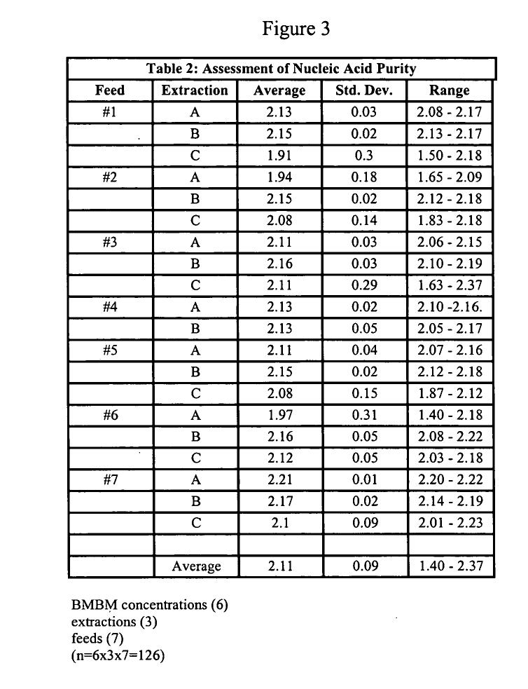

FIG. 4 is a table (Table 3) summarizing the effect of (1) RNase treatment; and (2) the type of feed and the concentration of bovine meat bovine meal (BMBM) on the detection of bovine mtDNA. RNAse treatment improved the B-mtDNA detection sensitivity and B-mtDNA detection consistency in Feed Nos. 3, 5, 6 and 7. B-mtDNA was detected in Feed Nos. 1 and 2 samples spiked with 0.10% BMBM. B-mtDNA was detected in Feed Nos. 1, 2 and 7 samples spiked with 0.1% BMBM. B-mtDNA was detected in Feed No. 1 samples spiked with 0.05% BMBM. B-mtDNA was detected in all feeds treated with RNAse and spiked with 0.02% BMBM. With the exception of Feed No. 3, B-mtDNA was detected in all feeds spiked with 0.1% BMBM.

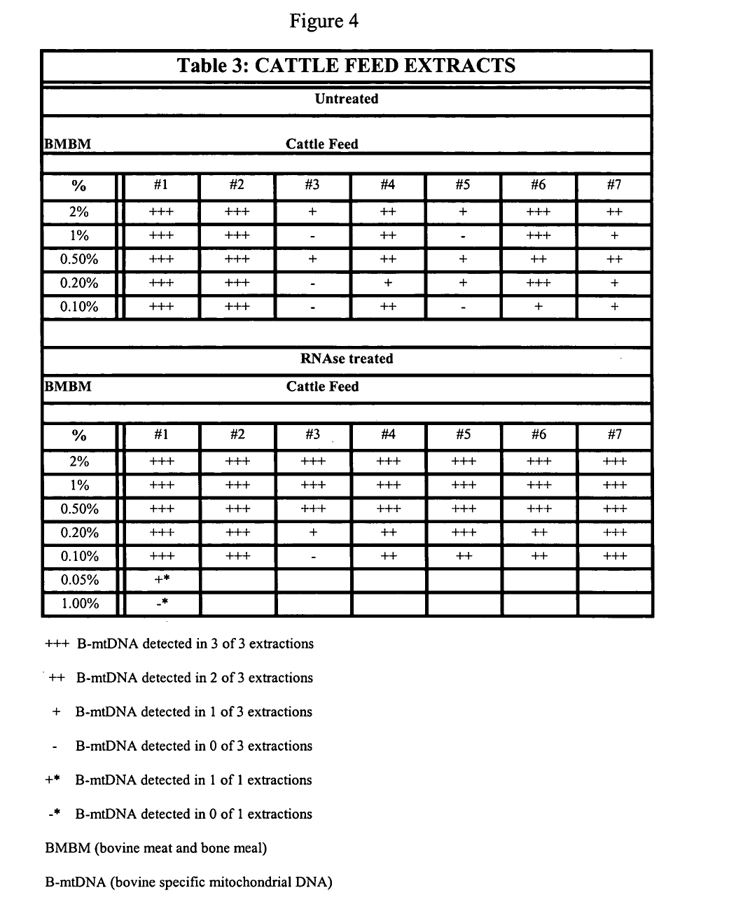

FIG. 5 is a table (Table 4) summarizing the effect of RNase treatment on the number of false negative results. Overall, RNAse treatment decreased false negative results 75%, (42/105 to 10/105). False negative results in feed samples containing the highest concentrations of BMBM (2%, 1% and 0.5%) decreased 100% (22/63 to 0/63). False negative results in feed samples containing the lowest concentrations of BMBM (0.2% and 0.1%) decreased by 50% (20/42 to 10/42). All feed samples containing 0% BMBM were negative.

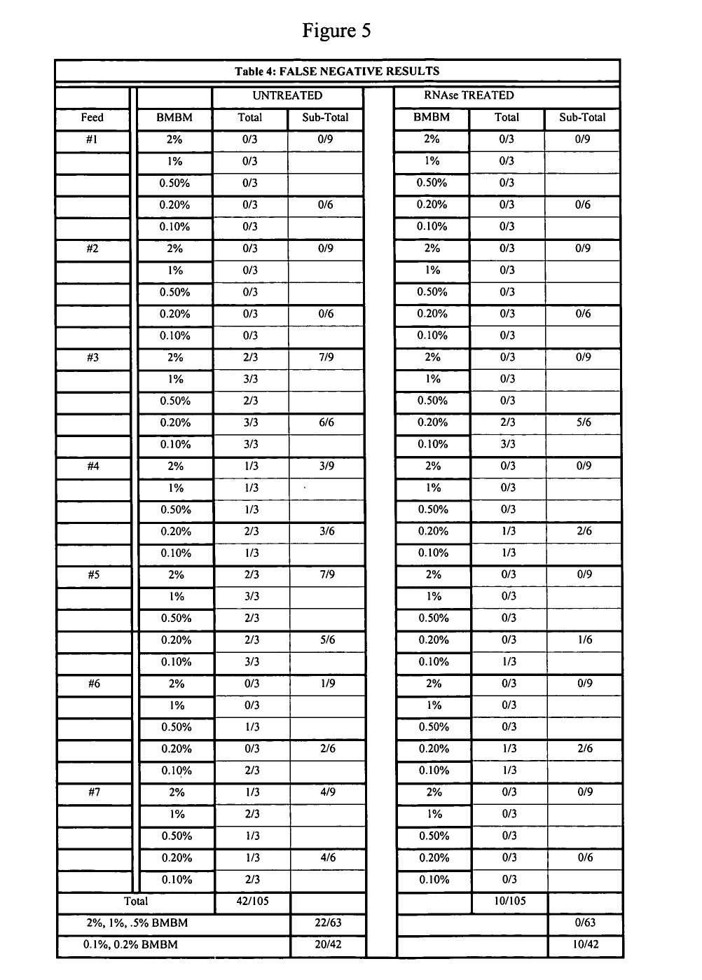

FIG. 6 shows detection of and differentiation between bovine, sheep, and goat species DNA in a single PCR reaction using a set of FRET probes (SEQ ID NOS:13 and 14) and primers (SEQ ID NOS:11 and 12) designed so that the DNA from all three species of ruminants would amplify, and the probes would bind to all three amplicons but with varying degrees of homology. The FRET probes bind to bovine target sequence with 100% homology, goat target sequence with 93% homology and sheep target sequence with 88% homology. The differences in homology result in three distinct melting curve temperatures (Tm), each corresponding to bovine, goat, or sheep species.

FIG. 7 shows data comparing a PCR-based method and an antibody-based method for detecting the presence of bovine dried blood (BDB) and bovine meat and bone meal (BMBM) in five representative cattle feeds. Results shown are the results of triplicate assays. All non-spiked feeds were negative with both methods.

FIG. 8 shows data demonstrating PCR reaction efficiencies of bovine DNA standard serially diluted into DNA extract from a vaccine sample.

### DETAILED DESCRIPTION OF THE INVENTION

I. Introduction

The present invention provides methods and kits for amplifying, measuring and/or detecting ruminant DNA in a sample (e.g., of an animal feed, an animal feed component, a cosmetic, a nutriceutical, a vaccine, a colloidal infusion fluid, or combinations thereof). In some embodiments, the invention provides methods for amplifying, measuring and/or detecting ruminant DNA in animal feed or animal feed components. The present invention is based on the surprising discovery that RNA present in a sample (e.g., a sample such as an animal feed, a cosmetic, a nutriceutical, or a vaccine that is being tested for the presence of ruminant DNA) interferes with amplification reactions for detecting ruminant DNA in the sample. The inventors have discovered that treatment of nucleic acids from samples with RNase improves the consistency and sensitivity of amplification reactions for detecting ruminant DNA. In particular, the inventors have discovered that treatment of nucleic acids from samples (e.g., samples being tested for the presence of ruminant DNA) with RNase reduces the incidence of false negatives when such nucleic acids are subjected to amplification reactions to detect ruminant DNA.

II. Definitions

A “sample” as used herein refers to a sample of any source which is suspected of containing ruminant polypeptides or nucleic acids encoding a ruminant polypeptide. These samples can be tested by the methods described herein and include, e.g., ruminant feed, pet food, cosmetics, human food, nutraceuticals, vaccines, or colloidal infusion fluids. A sample can be from a laboratory source or from a non-laboratory source. A sample may be suspended or dissolved in liquid materials such as buffers, extractants, solvents and the like. Samples also include animal and human body fluids such as whole blood, blood fractions, serum, plasma, cerebrospinal fluid, lymph fluids, milk; and biological fluids such as cell extracts, cell culture supernatants; fixed tissue specimens; and fixed cell specimens.

“Ruminant” as used herein refers to a mammal with having a stomach divided into multiple compartments (i.e., a rumen, a reticulum, an omasum, and an abomasum) and capable of digesting cellulose. Examples of ruminants include, e.g., cows, sheep, goats, deer, elk, buffalo, bison, llamas, alpacas, dromedaries, camels, yaks, reindeer, giraffes and the like.

“Animal feed” and “animal feed component” as used herein refers to any composition or portion thereof that supplies nutrition to an animal. General components of animal feed include, for example, protein, carbohydrate, and fat. Specific components of animal feed include, for example, corn, beef tallow, blood and/or fractions thereof, milk and/or fractions thereof, molasses/sugar (e.g., raw or processed sugar, molasses from beets, sugar cane and citrus, and combinations thereof), carrots, candy bars, grains (e.g., wheat, oats, barley, triticale, rice, maize/corn, sorghum, rye, and combinations thereof), processed grain fractions (e.g., pollard, bran, millrun, wheat germ, brewers grain, malt combings, biscuits, bread, hominy, semolina, and combinations thereof), pulses/legumes (e.g., succulent or mature dried seed and immature pods of leguminous plants, including for example, peas, beans, lentils, soya beans, and lupins, and combinations thereof), oil seeds (e.g., cotton seed, sunflower seed, safflower seed, rape/canola seed, linseed, and sesame seed, and combinations thereof); plant protein meals (e.g., oilseed meals, peanut meal, soya bean meal, copra meal, palm kernel meal, and combinations thereof); fruit by-products (e.g., citrus pulp, pineapple pulp, pome fruit pomace, grape marc, grape pomace, and combinations thereof), pasture (e.g., grass and legume pastures and mixed grass/legume pastures), fodder (e.g., seeds, hay, silage and straw of legumes, grasses and cereals, sugar cane tops, and combinations thereof), forage (e.g., cereal forage, oilseed forage, legume forage, , and combinations thereof), alfalfa (e.g., fresh, dried, mid bloom, and combinations thereof), barley grain, dried beet pulp, bluegrass, brewer's grains (e.g., wet, dried, and combinations thereof), Brome grass, Late Brome grass hay, Citrus pulp (e.g., dried, silage, and combinations thereof), clover (e.g., hay, silage, and combinations thereof), coconut meal, corn (e.g., cobs, ears, grain, silage, and combinations thereof), corn gluten feed, cottonseed (e.g., hulls, whole, meal, and combinations thereof), dried distiller's grain, fish meal, hominy feed, lamb meal, Lespedeza (e.g., fresh, hay, and combinations thereof), linseed meal, meat and bone meal (e.g., from cattle, sheep, goats, poultry, and combinations thereof), milk (fresh, dried, skimmed, and combinations thereof), millet, napier grass, orchard grass, peanut meal; natural sausage casings, foods containing “binders” comprising bovine collagen. Animal feed can also include supplemental components, such as, for example, minerals, vitamins, and nutraceuticals. Animal feed includes, for example, cattle feed, sheep feed, goat feed, dog feed, cat feed, deer feed, elk feed, and the like. Animal feed and animal feed components are understood to be compositions that do not normally contain ruminant DNA.

“Animals” or “animal” as used herein refers to any vertebrate organism. Animals include mammals, avians, amphibians, reptiles, ruminants, primates (e.g., humans, gorillas, and chimpanzees). Animals include domesticated animals (e.g., cattle, sheep, goats, pigs, chickens, ducks, turkeys, geese, quail, guinea hens, cats, and dogs) as well as undomesticated animals (e.g., elk, deer, reindeer, and giraffes). Animals may in the wild (i.e., in their native environments) or may be maintained in zoological parks. Other animals within the definition used herein include, for example, elephants, rhinoceroses, hippopotami, lions, tigers, bears, cougars, pumas, bobcats, and the like.

A “cosmetic” or “cosmeceutical” as used herein refers to any compound intended to be rubbed, poured, sprinkled, or sprayed on, introduced into, or otherwise applied to the human body for cleansing, beautifying, promoting attractiveness, or altering the appearance. Exemplary types of cosmetics include, e.g., skin conditioning agents, emollients, binders, and hair and nail conditioning agents. Exemplary cosmetics include, e.g., skin moisturizers (including, e.g., body lotions, skin lotions, and anti-wrinkle creams), skin cleansers, acne care products (including, e.g., skin moisturizers, skin cleansers, skin toners, and concealers) perfumes, lip moisturizers, lip balms, lipsticks, fingernail polishes, eye and facial makeup preparations, shampoos, hair conditioners, permanent waves, hair dyes, toothpastes, collagen implants, and deodorants, as well as any material intended for use as a component of a cosmetic product.

A “nutraceutical” as used herein refers to any substance that is a food or a part of a food and provides medical or health benefits, including the prevention and treatment of disease. Nutraceuticals include, e.g., isolated nutrients, dietary supplements and specific diets to genetically engineered designer foods, herbal products, and processed foods such as cereals, soups and beverages, a product isolated or purified from foods, and generally sold in medicinal forms not usually associated with food and demonstrated to have a physiological benefit or provide protection against chronic disease. Nutraceuticals also include any food that is nutritionally enhanced with nutrients, vitamins, or herbal supplements. Exemplary nutraceuticals include nutritional supplements such as, e.g., amino acids (including, e.g., Tyrosine, Tryptophan); oils and fatty acids (including, e.g., Linoleic acid and Omega 3 oils); minerals/coenzymes/trace elements (including, e.g., Iron, Coenzyme Q10, Zinc); vitamins (including, e.g., Ascorbic acid, Vitamin E); Protein (whey) powders/drinks; plant based/herbs (including, e.g., alfalfa, phytonutrients, saw palmetto); Herbal and Homeopathic remedies (including, e.g., Leopard's bane, St John's wort; Colitis treatments (including, e.g., those that contain bovine colostrums such as enemas); arthritis treatments (including, e.g., those that contain bovine glucosamine-chondroitin); joint cartilage replacements (including, e.g., those that contain bovine cartilage); digestive aids (bile salts, garlic oils); and weight management products (including, e.g., those that contain bovine proteins such as collagen, gelatin and whey protein).

A “vaccine” as used herein refers to a preparation comprising an infectious or immunogenic agent which is administered to stimulate a response (e.g., and immune response) that will protect the individual to whom it is administered from illness due to an infectious agent. Individuals to whom vaccines may be administered include any animals as defined herein. Vaccines include therapeutic vaccines given after infection and intended to reduce or arrest disease progression as well as preventive (i.e., prophylactic) vaccines intended to prevent initial infection. Infectious agents used in vaccines may be whole-killed (inactive), live-attenuated (weakened) or artificially (e.g. recombinantly) manufactured bacteria, viruses, or fungi. Exemplary vaccines include, e.g., E. coli Bacterin J5 strain (Upjohn), UltraBac 7 (Clostridum Chauvoei-Septicum-Novyi-Sordellii-Perfringens Types C&D Bacterin-Toxoid) (Pfizer), Spirovav (Leptospira Hardjo Bacterin) (Pfizer), Leptoferm-5 (Leptospira Canicola-Grippotyphosa-Hardjo-Icterohaemorrhagiae-Pomona Bacterin) (Pfizer), ScourGuard 3 (Bovine Rota-Coronavirus-Killed Virus) Clostridium Perfringens Type C-E. coli Bacterin-Toxoid) (Pfizer), Bovi-Shield Gold (Bovine Rhinotracheitis-Virus Diarrhea-Parainfluenza-Respiratory Syncytial Virus Vaccine Modified Live Virus) Leptospira Canicol-Grippotyphosa-Hardjo-Icterohaemorrhagiae-Pomona Bacterin (Pfizer), Defensor 3 Rabies Vaccine killed virus (Pfizer), and Vanguard Plus 5 Canine Distemper-Adenovirus Type 2-Coronavirus-Parainfluenza-Parvovirus Vaccine Modified Live killed Virus Leptospira Bacterin (Pfizer).

A “colloidal infusion fluid” as used herein refers to a fluid that when administered to a patient, can cause significant increases in blood volume, cardiac output, stroke volume, blood pressure, urinary output and oxygen delivery. Exemplary colloidal infusion fluids include, e.g., plasma expanders. Plasma expanders are blood substitute products useful for maintaining patients' circulatory blood volume during surgical procedures or trauma care hemorrhage, acute trauma or surgery, bums, sepsis, peritonitis, pancreatitis or crush injury. Exemplary plasma expanders include, e.g., albumin, gelatin-based products such as Gelofusine®, and collagen-based products. Plasma expanders may be derived from natural products or may be recombinantly produced.

“RNase” as used herein refers to an enzyme that catalyzes the hydrolysis (i.e., degradation) of ribonucleic acid. Suitable RNases include, for example, RNase A, RNase B, RNase D, RNase E, RNase H, RNase I, RNase P, RNase S, RNase T, and RNase V. RNases hydrolyze RNA in both single- and double-stranded form, and recognize particular ribonucleic acid residues. For example, RNase A cleaves 3′ of single-stranded C and U residues; RNase D hydrolyzes duplex RNA; RNase H specifically degrades the RNA in RNA:DNA hybrids; RNase I preferentially degrades single stranded RNA into individual nucleoside 3′ monophosphates by cleaving every phosphodiester bond; RNase T1 cleaves 3′ of single-stranded G residues; and RNase V1 cleaves base-paired nucleotides.

“PCR inhibitor” as used herein refers to any compound that affects a PCR amplification process, i.e., by interfering with any portion the amplification process itself or by interfering with detection of the amplified product. The PCR inhibitor may physically, i.e., mechanically interfere with the PCR reaction or detection of the amplified product. Alternatively, the PCR inhibitor may chemically interfere with the PCR reaction or detection of the amplified product.

An “amplification reaction” refers to any chemical reaction, including an enzymatic reaction, which results in increased copies of a template nucleic acid sequence. Amplification reactions include polymerase chain reaction (PCR) and ligase chain reaction (LCR) (see U.S. Pat. Nos. 4,683,195 and 4,683,202; PCR Protocols: A Guide to Methods and Applications (Innis et al., eds, 1990)), strand displacement amplification (SDA) (Walker, et al. Nucleic Acids Res. 20(7):1691 (1992); Walker PCR Methods Appl 3(l):1 (1993)), transcription-mediated amplification (Phyffer, et al, J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), rolling circle amplification (RCA) (Lisby, Mol. Biotechnol. 12(1):75 (1999)); Hatch et al., Genet. Anal. 15(2):35 (1999)) and branched DNA signal amplification (bDNA) (see, e.g., Iqbal et al., Mol. Cell Probes 13(4):315 (1999)).

“Amplifying” refers to submitting a solution to conditions sufficient to allow for amplification of a polynucleotide if all of the components of the reaction are intact. Components of an amplification reaction include, e.g., primers, a polynucleotide template, polymerase, nucleotides, and the like. Thus, an amplifying step can occur without producing a product if, for example, primers are degraded.

“Detecting” as used herein refers to detection of an amplified product, i.e., a product generated using the methods known in the art. Suitable detection methods are described in detail herein. Detection of the amplified product may be direct or indirect and may be accomplished by any method known in the art. The amplified product can also be measured (i.e., quantitated) using the methods known in the art.

“Amplification reagents” refer to reagents used in an amplification reaction. These reagents can include, e.g., oligonucleotide primers; borate, phosphate, carbonate, barbital, Tris, etc. based buffers (see, U.S. Pat. No. 5,508,178); salts such as potassium or sodium chloride; magnesium; deoxynucleotide triphosphates (dNTPs); a nucleic acid polymerase such as Taq DNA polymerase; as well as DMSO; and stabilizing agents such as gelatin, bovine serum albumin, and non-ionic detergents (e.g. Tween-20).

The term “primer” refers to a nucleic acid sequence that primes the synthesis of a polynucleotide in an amplification reaction. Typically a primer comprises fewer than about 100 nucleotides and preferably comprises fewer than about 30 nucleotides. Exemplary primers range from about 5 to about 25 nucleotides. The “integrity” of a primer refers to the ability of the primer to primer an amplification reaction. For example, the integrity of a primer is typically no longer intact after degradation of the primer sequences such as by endonuclease cleavage.

A “probe” or “oligonucleotide probe” refers to a polynucleotide sequence capable of hybridization to a polynucleotide sequence of interest and allows for the detecting of the polynucleotide sequence of choice. For example, “probes” can comprise polynucleotides linked to fluorescent or radioactive reagents, thereby allowing for the detection of these reagents.

The term “subsequence” refers to a sequence of nucleotides that are contiguous within a second sequence but does not include all of the nucleotides of the second sequence.

A “target” or “target sequence” refers to a single or double stranded polynucleotide sequence sought to be amplified in an amplification reaction. Two target sequences are different if they comprise non-identical polynucleotide sequences. The target sequences may be mitochondrial DNA or non-mitochondrial DNA. Suitable mitochondrial target sequences include, for example, cytochrome B, cytochrome C, 12S RNA, ATPase subunit 8, ATPase subunit 6, ATP synthetase, subunit 8, and subsequences, and combinations thereof.

The phrase “nucleic acid” or “polynucleotide” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Two nucleic acid sequences or polypeptides are said to be “identical” if the sequence of nucleotides or amino acid residues, respectively, in the two sequences is the same when aligned for maximum correspondence as described below. The term “complementary to” is used herein to mean all of a first sequence is complementary to at least a portion of a reference polynucleotide sequence.

Optimal alignment of sequences for comparison may be conducted by the local homology algorithm of Smith and Waterman Add. APL. Math. 2:482 (1981), by the homology alignment algorithm of Needle man and Wunsch J. Mol. Biol. 48:443 (1970), by the search for similarity method of Pearson and Lipman Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. The percent identity between two sequences can be represented by any integer from 25% to 100%. More preferred embodiments include at least: 25%, 30%, 35%, 40%, 45%, 50%, 55%, 60%, 65%, 70%, 75%, 80%, 85%, 90%, 95%, or 99%.

One example of an algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. Mixed nucleotides are designated as described in e.g. Eur. J. Biochem. (1985) 150:1.

III. Methods of the Invention

One embodiment of the present invention provides methods of amplifying, detecting, and/or measuring ruminant DNA in samples (e.g., ruminant feed, pet food, cosmetics, human food, and nutraceuticals). Target ruminant DNA sequences of particular interest include mitochondrial DNA sequences and non-mitochondrial DNA sequences. Suitable mitochondrial DNA sequences include, for example, sequences encoding: cytochrome c, cytochrome b, 12S RNA, ATPase subunit 8, ATPase subunit 6, ATP synthetase, subunit 8, and subsequences and combinations thereof.

A. RNase treatment

According to the methods of the invention, nucleic acids from the samples are contacted with an RNase under conditions (e.g., appropriate time, temperature, and pH) suitable for the RNase to degrade any RNA present in the animal feed, thus reducing and/or eliminating an inhibitor of the amplification reaction used to amplify ruminant DNA in the animal feed. Typically the RNase is contacted with the nucleic acid for about 15 to about 120 minutes, more typically for about 30 to about 90 minutes, even more typically for about 45 to about 75 minutes, most typically, for about 60 minutes. Typically, the RNase is contacted with the nucleic acid at about 30° C. to about 42° C., more typically at about 35° C. to about 40° C., most typically at about 37° C. Typically, the RNase is contacted with the nucleic acid at about pH 6.5 to about 8.0, more typically at about 6.8 to about 7.5, most typically at about pH 7.0. Typically, about 0.01 to about 1 μg RNase is contacted with the nucleic acid, more typically about 0.025 to about 0.5 μg RNase is contacted with the nucleic acid, more typically about 0.4 to about 0.25 μg RNase is contacted with the nucleic acid, most typically, about 0.05 μg RNase is contacted with the nucleic acid. In some embodiments, the RNase is heated to about 100° C. to destroy any contaminating DNase prior to contacting the RNase with the nucleic acid.

One of skill in the art will appreciate that the RNase can be contacted with the nucleic acid before, during, or after extraction of the nucleic acid from the animal feed. One of skill in the art will also appreciate that any RNase known in the art can be used in the methods of the invention. Suitable RNases include, for example, RNase A, RNase B, RNase E, RNase H, RNase I, RNase P, RNase S, RNase T, RNase V, and combinations thereof. Many RNases and combinations of RNases are available commercially. For example, DNase free-RNase from Roche Diagnostics Corporation (Catalog No. 1 119 915) can conveniently be used in the methods of the invention.

B. Nucleic Acid Extraction

Nucleic acids can be extracted from the sample using any method known in the art and/or commercially available kits. For example, guanidine isothiocyanate extraction as described in Tartaglia et al., J. Food Prot. 61(5):513-518 (1998); chelex extraction as described in Wang et al., Mol. Cell. Probes 14:1-5 (2000); extraction from Whatman paper as described in U.S. Pat. No. 5,496,562; extraction from cellulose based FTA filters as described in Orlandi and Lampe, J. Clin. Microbiology, 38(6): 2271-2277 (2000) and Burgoyne et al., 5th International Symposium on Human Identification, 1994 (Hoenecke et al., eds.) can be used to extract nucleic acids from the samples. In addition, the Neogen Kit (Neogen Catalog No. 8100), the Qiagen Stool Kit (Qiagen Catalog No. 51504), the Qiagen Plant Kit (Qiagen Catalog No. 69181), and Whatman FTA cards (e.g., Whatman Catalog Nos. WB120055; WB120056; WB120205; WB120206; WB120208; WB120210) can conveniently be used to extract nucleic acids from any sample.

In a preferred embodiment, cellulose based FTA cards are used to extract nucleic acid. The FTA cards typically comprise compounds that lyse cell membranes and denature proteins. Samples are applied to the FTA card and allowed to dry. DNA is captured within the matrix of the FTA cards and is stable at room temperature for up to 14 years. For extraction of nucleic acids for PCR analysis of the sample (e.g., animal feed, human food, a vaccine, a cosmetic, or a nutraceutical), a punch (e.g., a 1-2 mm punch) is taken from the FTA card and the FTA card is washed according to manufacturer's instructions. The washed punch can then either be placed directly into a PCR reaction or the DNA can be eluted from the punch using any method known in the art. Liquid samples can be applied directly to the card without pre-processing. More complex samples (e.g., solid samples) may require processing prior to application to the FTA card. Typically, about 1 μl to about 1000 μl, more typically about 2.5 to about 500 μl, more typically about 5 μl to about 250 μl, more typically about 7.5 μl to about 100 μl , most typically about 10 μl to 65 μl sample can be placed on the FTA card.

Basic texts disclosing the general methods of use in this invention include MOLECULAR CLONING: A LABORATORY MANUAL (Sambrook et al. eds. 3d ed. 2001); PCR PROTOCOLS: A GUIDE TO METHODS AND Applications (Innis et al., eds, 1990); GENE TRANSFER AND EXPRESSION: A LABORATORY MANUAL (Kriegler, 1990); and CURRENT PROTOCOLS IN MOLECULAR BIOLOGY (Ausubel et al., eds., 1994)).

C. Amplification Reaction Components
1. Oligonucleotides

The oligonucleotides that are used in the present invention as well as oligonucleotides designed to detect amplification products can be chemically synthesized, using methods known in the art. These oligonucleotides can be labeled with radioisotopes, chemiluminescent moieties, or fluorescent moieties. Such labels are useful for the characterization and detection of amplification products using the methods and compositions of the present invention.

Typically, the target primers are present in the amplification reaction mixture at a concentration of about 0.1 μM to about 1.0 μM, more typically about 0.25 μM to about 0.9 μM, even more typically about 0.5 to about 0.75 μM, most typically about 0.6 μM. The primer length can be about 8 to about 100 nucleotides in length, more typically about 10 to about 75 nucleotides in length, more typically about 12 to about 50 nucleotides in length, more typically about 15 to about 30 nucleotides in length, most typically about 19 nucleotides in length. Preferably, the primers of the invention all have approximately the same melting temperature. Typically, the primers amplify a sequence of ruminant DNA which exhibits high interspecies variation. Suitable target sequences include, for example, cytochrome B, cytochrome C, 12S RNA, ATPase subunit 8, ATPase subunit 6, ATP synthetase, subunit 8, and subsequences, and combinations thereof.
2. Buffer

Buffers that may be employed are borate, phosphate, carbonate, barbital, Tris, etc. based buffers. (See, U.S. Pat. No. 5,508,178). The pH of the reaction should be maintained in the range of about 4.5 to about 9.5. (See, U.S. Pat. No. 5,508,178. The standard buffer used in amplification reactions is a Tris based buffer between 10 and 50 mM with a pH of around 8.3 to 8.8. (See Innis et al., supra.).

One of skill in the art will recognize that buffer conditions should be designed to allow for the function of all reactions of interest. Thus, buffer conditions can be designed to support the amplification reaction as well as any subsequent restriction enzyme reactions. A particular reaction buffer can be tested for its ability to support various reactions by testing the reactions both individually and in combination.
3. Salt Concentration

The concentration of salt present in the reaction can affect the ability of primers to anneal to the target nucleic acid. (See, Inis et al.). Potassium chloride can added up to a concentration of about 50 mM to the reaction mixture to promote primer annealing. Sodium chloride can also be added to promote primer annealing. (See, Innis et al.).
4. Magnesium Ion Concentration

The concentration of magnesium ion in the reaction can affect amplification of the target sequence(s). (See, Innis et al.). Primer annealing, strand denaturation, amplification specificity, primer-dimer formation, and enzyme activity are all examples of parameters that are affected by magnesium concentration. (See, Innis et al.). Amplification reactions should contain about a 0.5 to 2.5 mM magnesium concentration excess over the concentration of dNTPs. The presence of magnesium chelators in the reaction can affect the optimal magnesium concentration. A series of amplification reactions can be carried out over a range of magnesium concentrations to determine the optimal magnesium concentration. The optimal magnesium concentration can vary depending on the nature of the target nucleic acid(s) and the primers being used, among other parameters.
5. Deoxynucleotide Triphosphate Concentration

Deoxynucleotide triphosphates (dNTPs) are added to the reaction to a final concentration of about 20 μM to about 300 μM. Typically, each of the four dNTPs (G, A, C, T) are present at equivalent concentrations. (See, Innis et al.).
6. Nucleic acid polymerase

A variety of DNA dependent polymerases are commercially available that will function using the methods and compositions of the present invention. For example, Taq DNA Polymerase may be used to amplify target DNA sequences. The PCR assay may be carried out using as an enzyme component a source of thermostable DNA polymerase suitably comprising Taq DNA polymerase which may be the native enzyme purified from Thermus aquaticus and/or a genetically engineered form of the enzyme. Other commercially available polymerase enzymes include, e.g., Taq polymerases marketed by Promega or Pharmacia. Other examples of thermostable DNA polymerases that could be used in the invention include DNA polymerases obtained from, e.g., Thermus and Pyrococcus species. Concentration ranges of the polymerase may range from 1-5 units per reaction mixture. The reaction mixture is typically between 15 and 100 μl.

In some embodiments, a “hot start” polymerase can be used to prevent extension of mispriming events as the temperature of a reaction initially increases. Hot start polymerases can have, for example, heat labile adducts requiring a heat activation step (typically 95° C. for approximately 10-15 minutes) or can have an antibody associated with the polymerase to prevent activation.
7. Other Agents

Additional agents are sometime added to the reaction to achieve the desired results. For example, DMSO can be added to the reaction, but is reported to inhibit the activity of Taq DNA Polymerase. Nevertheless, DMSO has been recommended for the amplification of multiple target sequences in the same reaction. (See, Innis et al. supra). Stabilizing agents such as gelatin, bovine serum albumin, and non-ionic detergents (e.g. Tween-20) are commonly added to amplification reactions. (See, Innis et al. supra).

D. Amplification

Amplification of an RNA or DNA template using reactions is well known (see, U.S. Pat. Nos. 4,683,195 and 4,683,202; PCR PROTOCOLS: A GUIDE TO METHODS AND APPLICATIONS (Innis et al., eds, 1990)). Methods such as polymerase chain reaction (PCR) and ligase chain reaction (LCR) can be used to amplify nucleic acid sequences of target DNA sequences directly from animal feed and animal feed components. The reaction is preferably carried out in a thermal cycler to facilitate incubation times at desired temperatures. Degenerate oligonucleotides can be designed to amplify target DNA sequence homologs using the known sequences that encode the target DNA sequence. Restriction endonuclease sites can be incorporated into the primers.

Exemplary PCR reaction conditions typically comprise either two or three step cycles. Two step cycles have a denaturation step followed by a hybridization/elongation step. Three step cycles comprise a denaturation step followed by a hybridization step followed by a separate elongation step. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 15 seconds.-2 minutes, an annealing phase lasting 10 seconds-2 minutes, and an extension phase of about 72° C. for 5 seconds-2 minutes.

In some embodiments, the amplification reaction is a nested PCR assay as described in, e.g., Aradaib et al., Vet. Sci. Animal Husbandry 37 (1-2): 13-23 (1998) and Aradaib et al., Vet. Sci. Animal Husbandry 37 (1-2): 144-150 (1998). Two amplification steps are carried out. The first amplification uses an “outer” pair of primers (e.g., SEQ ID NOS:7 and 10) designed to amplify a highly conserved region of the target sequence . The second amplification uses an “inner” (i.e., “nested”) pair of primers (e.g., SEQ ID NOS:8 and 9) designed to amplify a portion of the target sequence that is contained within the first amplification product.

Isothermic amplification reactions are also known and can be used according to the methods of the invention. Examples of isothermic amplification reactions include strand displacement amplification (SDA) (Walker, et al. Nucleic Acids Res. 20(7):1691 (1992); Walker PCR Methods Appl 3(1):1 (1993)), transcription-mediated amplification (Phyffer, et al., J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), and branched DNA signal amplification (bDNA) (see, e.g., Iqbal et al., Mol. Cell Probes 13(4):315 (1999)). In a preferred embodiment, rolling circle amplification (RCA) (Lisby, Mol. Biotechnol. 12(1):75 (1999)); Hatch et al., Genet. Anal. 15(2):35 (1999)) is used. Other amplification methods known to those of skill in the art include CPR (Cycling Probe Reaction), SSR (Self-Sustained Sequence Replication), SDA (Strand Displacement Amplification), QBR (Q-Beta Replicase), Re-AMP (formerly RAMP), RCR (Repair Chain Reaction), TAS (Transcription Based Amplification System), and HCS (hybrid capture system). Any amplification method known to those of skill in the art may be used with the methods of the present invention provided two primers are present at either end of the target sequence.

E. Detection of Amplified Products

Any method known in the art can be used to detect the amplified products, including, for example solid phase assays, anion exchange high-performance liquid chromatography, and fluorescence labeling of amplified nucleic acids (see MOLECULAR CLONING: A LABORATORY MANUAL (Sambrook et al. eds. 3d ed. 2001); Reischl and Kochanowski, Mol. Biotechnol. 3(1): 55-71 (1995)). Gel electrophoresis of the amplified product followed by standard analyses known in the art can also be used to detect and quantify the amplified product. Suitable gel electrophoresis-based techniques include, for example, gel electrophoresis followed by quantification of the amplified product on a fluorescent automated DNA sequencer (see, e.g., Porcher et al., Biotechniques 13(1): 106-14 (1992)); fluorometry (see, e.g., Innis et al., supra), computer analysis of images of gels stained in intercalating dyes (see, e.g., Schneeberger et al., PCR Methods Appl. 4(4): 234-8 (1995)); and measurement of radioactivity incorporated during amplification (see, e.g., Innis et al., supra). Other suitable methods for detecting amplified products include using dual labeled probes, e.g., probes labeled with both a reporter and a quencher dye, which fluoresce only when bound to their target sequences; and using fluorescence resonance energy transfer (FRET) technology in which probes labeled with either a donor or acceptor label bind within the amplified fragment adjacent to each other, fluorescing only when both probes are bound to their target sequences. Suitable reporters and quenchers include, for example, black hole quencher dyes (BHQ), TAMRA, FAM, CY3, CY5, Fluorescein, HEX, JOE, LightCycler Red, Oregon Green, Rhodamine, Rhodamine Green, Rhodamine Red, ROX, TAMRA, TET, Texas Red, and Molecular Beacons.

The amplification and detection steps can be carried out sequentially, or simultaneously. In a preferred embodiment, RealTime PCR is used to detect target sequences. For example, in a preferred embodiment, Real-time PCR using SYBR® Green I can be used to amplify and detect the target nucleic acids (see, e.g., Ponchel et al., BMC Biotechnol. 3:18 (2003)). SYBR® Green I only fluoresces when bound to double-stranded DNA (dsDNA). Thus, the intensity of the fluorescence signal depends on the amount of dsDNA that is present in the amplified product. Specificity of the detection can conveniently be confirmed using melting curve analysis.

In another preferred embodiment, FRET probes and primers can be used to detect the ruminant DNA. One of skill in the art will appreciate that the primers and probes can conveniently be designed for use with the Lightcycler system (Roche Molecular Biochemicals). For example, a single set of primers (e.g., SEQ ID NOS:11 and 12) and probes (SEQ ID NOS:13 and 14) can conveniently be designed so that the DNA from multiple species of ruminants (e.g., cattle, goat, sheep, elk, deer, and the like) would amplify, and the probes would bind to all amplicons but with varying degrees of homology. The differences in homology result in distinct melting curve temperatures (Tm), each corresponding to an individual ruminant species.

IV. Kits of the Invention

The present invention also provides kits for amplifying ruminant DNA. Such kits typically comprise two or more components necessary for amplifying ruminant DNA. Components may be compounds, reagents, containers and/or equipment. For example, one container within a kit may contain a first set of primers, e.g., SEQ ID NOS:1 and 2; 3 and 4; or 5 and 6 and another container.within a kit may contain a second set of primers, e.g., SEQ ID NOS:1 and 2; 3 and 4; or 5 and 6. In addition, the kits comprise instructions for use, i.e., instructions for using the primers in amplification and/or detection reactions as described herein.

The kits may further comprise any of the extraction, amplification, detection reaction components or buffers described herein. The kits may also comprise suitable RNases (e.g., RNase A, RNase B, RNase D, RNase E, RNase H, RNase I, RNase P, RNase S, RNase T, RNase V, and combinations thereof) for use in the methods of the invention.

### EXAMPLES

The embodiments of the present invention are further illustrated by the following examples. These examples are offered to illustrate, but not to limit the claimed invention.

### Example 1

### Materials and Methods

Preparation of cattle feed: Seven representative cattle feed samples were ground to a fine powder in a Wiley mill (Arthur H Thomas Co, Swedesboro, N.J., model 3375-E10) following official methods of analysis (see, e.g., JAOC, 16th Edition published by AOAC, International Suite 400, 2200 Wilson Blvd., Arlington Va. 22201 1995, §§ 965.16 and 950.02). The seven feeds comprised the following components:

Feed No. 1 (“Finishing” Ration I): 80% concentrate (corn), 20% roughage without molasses and bovine tallow;

| Ingredient | % Dry Matter |
|---|---|
| Alfalfa haylage | 4.63 |
| Alfalfa hay | 12.96 |
| Wheatlage | 3.70 |
| Corn silage | 25.74 |
| Almond hulls | 4.63 |
| Citrus pulp (wet) | 3.70 |
| Corn-flaked | 18.15 |
| Cottonseed (whole) | 8.33 |
| Soybean meal | 4.44 |
| Canola meal | 2.78 |
| Bypass soybean meal | 4.63 |
| Bypass protein mix (fish/blood) | 1.48 |
| Mineral mix | 3.89 |

Feed No. 2 (“Finishing” Ration II): 80% concentrate (corn), 20% roughage with molasses and bovine tallow

| Ingredient | % Dry Matter |
|---|---|
| Alfalfa haylage | 4.63 |
| Alfalfa hay | 12.96 |
| Wheatlage | 3.70 |
| Corn silage | 25.74 |
| Almond hulls | 4.63 |
| Citrus pulp (wet) | 3.70 |
| Corn-flaked | 18.15 |
| Cottonseed (whole) | 8.33 |
| Soybean meal | 4.44 |
| Canola meal | 2.78 |
| Bypass soybean meal | 4.63 |
| Bypass protein mix (fish/blood) | 1.48 |
| Mineral mix | 3.89 |
| Fat (tallow beef) | 0.5 |
| Molasses | 0.43 |

Feed No. 3 (“Starter” Ration): 40% concentrate (corn), 60% roughage;

| Ingredient | % Dry Matter |
|---|---|
| Alfalfa hay | 17.96 |
| Oat hay | 13.13 |
| Corn silage | 27.63 |
| Wheatlage | 10.36 |
| Mineral | 6.04 |
| Canola meal | 11.05 |
| Citrus pulp (wet) | 5.18 |
| Corn-flaked | 5.64 |

No. 4 (“Grower” Ration): 60% concentrate (corn) and 40% roughage and Dairy Feed Samples;

| Ingredient | % Dry Matter |
|---|---|
| Ground Corn | 38.6 |
| Cottonseed meal | 1.4 |
| Alfalfa hay | 12.0 |
| Corn silage | 44.0 |
| Mineral mix | 4.0 |

Feed No. 5 (“Adult Low Milk Production” Ration):

| Ingredient | % Dry Matter |
|---|---|
| Alfalfa haylage | 7.14 |
| Alfalfa hay | 15.48 |
| Corn silage | 28.57 |
| Almond hulls | 2.86 |
| Citrus pulp (wet) | 4.29 |
| Corn-flaked | 16.67 |
| Cottonseed (whole) | 9.52 |
| Soybean meal | 4.76 |
| Bypass soybean meal | 4.29 |
| Mineral mix | 4.76 |
| Molasses/fat blend | 1.67 |

Feed No. 6 (3-6 Month Calf Ration):

| Ingredient | % Dry Matter |
|---|---|
| Wheat straw | 11.49 |
| Alfalfa haylage | 17.01 |
| Milk cow refusal* | 22.99 |
| Wheatlage | 32.18 |
| Canola meal | 2.30 |
| Citrus pulp (wet) | 4.60 |
| Corn-flaked | 6.90 |
| Mineral | 2.53 |

*Milk cow refusal is the feed not consumed from the high production ration (finishing ration) that is gathered up and mixed with this heifer ration

Feed No. 7 (Commercial Calf Weaning Ration):

| Ingredient | % Dry Matter |
|---|---|
| Alfalfa hay | 16.09 |
| Corn silage | 30.65 |
| Wheatlage | 19.16 |
| Soybean meal | 9.96 |
| Corn-flaked | 19.16 |
| Mineral | 4.98 |

To confirm the absence of trace amounts of bovine products in the feeds, all feeds (unspiked and indicated as containing 0% bovine meat and bone marrow “BMBM”) were analyzed at the same time as the same feed spiked with rendered meat and bone meal. Rendered bovine meat and bone meal (BMBM) was mixed with the above seven feeds to produce feeds containing 2%, 1%, 0.5%, 0.2%, and 0.1% BMBM wt/wt. An unspiked sample of each feed (0% BMBM) was included as a negative control. One cattle feed (Feed 1) was selected to contain 0.05% and 0.01% BMBM and extracted only once.

DNA Extraction and Analysis with Qiagen Kit: Since it addressed the presence of PCR inhibitors in the samples, we chose the Qiagen Stool Kit (QIamp DNA Stool Mini Kit catalogue 51504 Qiagen Inc Valencia, Calif.) for our extractions. Using standard sampling procedures, non-specific DNA was extracted using minor modifications of the Qiagen Stool Kit protocol (see, e.g., J. Official Analy. Chem., §§ 965.16 and 950.02 (Assoc. Official Analy. Chem. 16th ed. (1995)). Briefly, the amount of reagent for digestion was increased to compensate for the adsorptive qualities of the powdered feed and only 100 μL was used for elution. The positive control was bovine mitochondrial DNA (B-mtDNA) extracted from BMBM using the Qiagen Stool kit; the negative controls were the feeds that were not spiked with BMBM (0% BMBM).

DNA Extraction and Analysis with Neogen Kit: DNA extraction was performed on spiked cattle feeds and run according to the instructions in the Neogen kit (Neogen Corporation, Lansing, Mich., AgriScreen for Ruminant Feed, catalogue 8100). Prior to PCR, the extracted product of the spiked and non-spiked cattle feeds was quantitated and assessed for purity. DNA was quantified using a fluorometer (Hoefer Pharmacia Biotech, San Francisco, Calif., model, TK-0-100). DNA purity (i.e., the 260/280 nm ratio) was measured using a spectrophotometer (Amersham Biosciences, San Francisco, Calif., model Ultraspec 2100). In one experiment, aliquots of selected extracts were placed in a boiling water bath for 10 minutes. DNA purity was further investigated by digestion of three selected samples with RNAse (DNA free RNAse—Roche Diagnostics Corporation Indianapolis, Ind., Catalogue 1 119 915) whereby 0.05 ug of RNAse was added to 10 μl of the extracted material and incubated at 37° C. for 60 minutes. The samples were then incubated at 95° C. for 10 minutes to inactivate the RNAse, then co-electrophoresed with the untreated extracts (1.2% agarose, containing ethidium bromide at 60 V for 50 minutes) using a DNA marker for comparison (Invitrogen 100 bp DNA Ladder, catalogue 10380, Carlsbad, Calif.). All cattle feed extracts were digested with RNAse as above and PCR performed on the untreated and RNAse treated extracts using the following PCR protocol.

PCR: Fluorescent PCR using hybridization probes and a Human DNA (HDNA) Control Kit (Roche, Applied Sciences, Indianapolis, Ind.) was performed on all seven feed samples containing 0% BMBM. The 18 μl reaction mixture contained 4 mM MgCl₂ beta-globin primer, LC Red 640 or LC Red 705, and the hybridization probes (Roche Applied Sciences). The tested feed was added to the reaction mixture in a ratio of 1:3.8 compared to PCR grade water added. Concentrations of 3 pg, 30 pg, 300 pg, 3 ng, and 30 ng of the Human Control Kit DNA were added in 2 μl increments as template DNA. The thermal settings used were: a denaturing step at 95° C. for 30 seconds; followed by 45 cycles at 95° C. for 0 seconds, 55° C. for 10 seconds, and 72° C. for 5 seconds; and a cooling period at 40° C. for 30 seconds. PCR grade water served as negative controls for each set. Separately, a set of controls was run in which no feed was added to the reaction mixture.

### Example 2

### Identification of RNA as a Contaminant Which Inhibits PCR Amplification of Ruminant DNA in Cattle Feeds

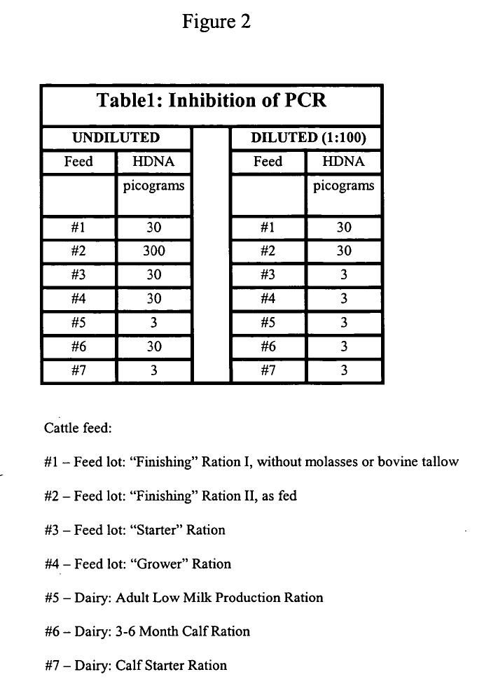

Assays using Human DNA as an internal PCR control indicated that PCR-inhibiting substances were present in the extracted product of cattle feeds. Inhibition was indicated by minimum picogram amounts of HDNA detected: (FIG. 2: Table 1). Minimum picogram amounts of HDNA varied one hundred fold among the seven undiluted cattle feed extracts. Diluting the extracts (1:100) increased the amplification of the detected HDNA. The minimum detection level was improved in Feed Nos. 2, 3, 4, and 6 by 10 fold; while the minimum detection level for Feed Nos. 1, 5, and 7 was unchanged. The addition of known amounts of an internal control such as HDNA for each feed sample enables detection of any inhibiting substances and interpretation of negative results. The difference in the detection levels of HDNA of the undiluted and diluted extracted products of the different cattle feeds confirms the presence of inhibiting substances which could potentially be diluted out.

A commercial immunoenzyme based test (Neogen) for ruminant contaminants in the feeds was also used. The Neogen test was unable to detect the spiked bovine product at a level lower than 1%, and in only one of the seven feeds. More particularly, the Neogen test was positive for B-mtDNA in only one feed spiked with 1% BMBM. In comparison, by PCR, we were able to detect B-mtDNA in the RNAse treated extracts in all samples spiked with 0.2% BMBM and with the exception of Feed No. 3 we were able to detect N-mtDNA in all cattle feeds spiked with 0.1% BMBM. We detected B-mtDNA in Feed 1 spiked with 0.05% BMBM. It is likely that B-mtDNA would be detected in other 0.5% BMBM-spiked feeds low in inhibitors (e.g., Feed Nos. 2 and 7.) Thus, our PCR assay has greater sensitivity than the detection limits of the Neogen kit.

Basic characterization of the inhibitory substance was undertaken. The inhibiting substances were first suspected to be enzymatic and/or proteinaceous in nature, however this possibility was excluded by the evidence that boiling had no effect on amplification of the extracted nucleic acids.

Measurements of the 260/280 nm ratio (average 2.11) of the extracted nucleic acids indicated that the nucleic acids were contaminated with RNA. The RNA contamination of the nucleic acids was confirmed by RNAse digestion of extracts and co-electrophoresis of the untreated and treated samples. A band of molecular weight below 2,000 bp suggests degraded DNA. Although DNA quantitation is preferably made with a fluorometer which detects only DNA; a spectrophotometer reading at 260 nm measures both DNA and RNA. The nucleic acid measurements (spectrophotometric 260 nm) were 10 to 40 times greater than the fluorometric DNA quantitations. This excessive amount of contaminating RNA measured in many of the extracts may interfere within the amplification reaction by mechanical means alone, i.e., by physical interference with the amplification reaction components. Interference caused by the presence of degraded DNA would generally lead to false positive results, however, we did not encounter any throughout this trial. Another possible explanation is that that the degraded DNA represented some of the target DNA, thus decreasing the B-mtDNA below the amount necessary for amplification. This may have contributed to the false negative results seen in the lower concentrations of 0.2% and 0.1% BMBM seen in RNAse treated cattle Feed Nos. 3, 4, 5, 6, and 7. An extraction process in which DNA integrity is better preserved and treatment of the cattle feeds with RNAse prior to column purification and concentration could theoretically increase the amount of B-mtDNA in the eluate and further improve the detection level.

Thus, we have confirmed the presence of PCR-inhibiting substances extracted simultaneously with non-specific DNA from seven representative types of cattle feed. Moreover, we have characterized and identified RNA as a major inhibitory substance.

### Example 3

### Amplification and Detection of Ruminant DNA in Multiple Animal Feeds and Feed Components

Fluorescent PCR using the Lightcycler (Roche Applied Sciences, Indianapolis, Ind.) was performed on all seven representative feeds containing 2%, 1%, 0.5%, 0.2%, 0.1%, and 0% bovine meat and bone meal (BMBM). Each of the untreated and RNAse treated samples were run at the same time. The high yield of mtDNA available from mammalian cells, the high mutation rate of mtDNA, and the genetic conservation of mtDNA make mitochondrial DNA highly suitable for use as target sequences specific for ruminant DNA, e.g., cattle DNA (see, e.g., Robin and Wong, J. Cell Physiol. 136:507-13 (1988) and Saccone et al., Gene 261:153-9 (2000).). Primers CSL1 and CSR2 amplify a 283 bp product: CSL1 B GAATTTCGGTTCCCTCCTG and CSR2 B GGCTATTACTGTGAGCAGA. A volume of 5 μL of extracted feed DNA was added to a 15 μL reaction containing 3.5 mM MgCl₂, 0.6 mM of each primer, and SYBR® Green I fluorescent dye. The thermal settings used were: a denaturing step at 95° for 30 seconds; followed by 40 cycles at 95° for 0 seconds, 56° for 10 seconds, and 72° C. for 12 seconds; a melting period at 95° C. for 0 seconds, 65° C. for 10 seconds, and 95° for 0 seconds; and a cooling period at 40° C. for 60 seconds. PCR negative (DNAse/RNAse free water) and positive (BMBM) controls were run along with the feed samples.

Additionally, PCR was performed on the samples using goat specific primers that yield a 428 bp product: GSL1 B TCATACATATCGGACGACGT and GSR2 B CAAGAATTAGTAGCATGGCG. The 15 μl reaction mixture contained 3 mM MgCl₂, 0.8 mM of both primers, and Fast Start SYBR® Green I dye (Roche Applied Sciences). The thermal settings used were: a denaturing step at 95° C. for 10 min; 45 cycles at 95° C. for 10 seconds, 57° C. for 5 seconds, and 72° C. for 25 seconds; a melting period at 95° C. for 0 seconds, 65° C. for 15 seconds, and 95° C. for 0 seconds; and a cooling period at 40° C. for 30 seconds.

In addition, rendered products from five animal species commonly used in animal feeds were extracted using the Qiagen Stool kit. The products used were pig dried blood, fish meal, lamb meal, poultry meal, and cattle dried blood. Each of the seven cattle feed samples were spiked with 2% wt/wt of each product. They were subjected to extraction of non-specific DNA, treated with RNAse and run using cattle specific primers, CSL1 and CSR2, and BMBM as the positive PCR control. A volume of 5 μL template DNA (“unknown” sample) was added to a 15 μL reaction mixture containing 3.5 mM MgCl₂, 0.6 mM of each primer, and SYBR® Green I dye. The thermal settings used were: a denaturing step at 95° C. for 30 seconds; followed by 40 cycles at 95° C. for 0 seconds, 56° C. for 10 seconds, and 72° C. for 12 seconds; a melting period of 95° C. for 0 seconds, 65° C. for 10 seconds, and 95° C. for 0 seconds; and a cooling period at 40° C. for 60 seconds.

Amplification of B-mtDNA occurred in only three feeds, the same feeds in which B-mtDNA was detected at the lowest level, i.e., feeds spiked with 0.1% BMBM. The inability to detect the mtDNA from rendered products of other species, especially those of closely related ruminants demonstrates the advantages of highly specific primers in PCR technology. Lack of detection with bovine dried blood in 4 of the seven cattle feeds is explained by leukocytes being the only nucleic acid material present in whole blood, hence the low amount of B-mtDNA available in the dried blood product. The three positive bovine dried blood in cattle Feed Nos. 1, 2 and 7 were the same 3 feeds, which when spiked with BMBM, had the lowest detectable amount of B-mtDNA. This indicates that RNAse treatment in these feeds was completely successful and that low amounts of amplicon can still be detected if the extracted product also contains low amounts of inhibiting substances. The negative results obtained using goat primers also attests to the specific nature of the goat-specific primers especially in the case of mtDNA from closely related ruminant species.

Thus we have measured the effect of the removal of RNA in the detection of B-mtDNA using fluorescent PCR technology.

### Example 4

### Amplification and Detection of Ruminant DNA in Cattle Feed

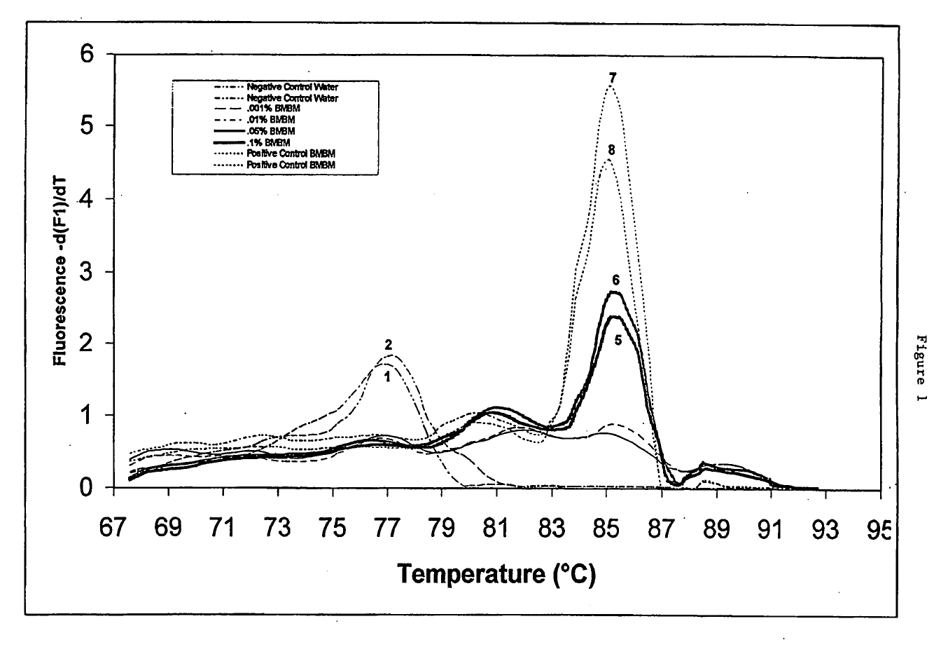

Cattle Feed 1 was “spiked” with 0.1%, 0.05 0.01% and 0.001% BMBM. The extracted products were run on the light cycler under the same conditions as the 7 RNAse treated feed samples. Melting curve analysis (FIG. 1) visually demonstrates amplification of target sequences. The melting temperature and cross-over point of the positive control was 85.28 and ¹9.05 respectively. Amplification products from feed samples containing 0.05% and 0.1% BMBM both had the same (85.28) melting temperature and had cross-over points of 25.67 and 24.96 respectively. The same extracted products were run on gel electrophoresis (1.2% agarose, containing ethidium bromide, at 60 V for 50 minutes). A DNA ladder (Invitrogen 100 bp Ladder, catalogue 10380, Carlsbad, Calif.) was used for comparison.

Cattle feeds were spiked with predetermined amounts of bovine meat and bone meal (BMBM). The extracted product was treated with RNAse and bovine specific mitochondrial DNA (B-mtDNA) and amplified with fluorescent lightcycler technology. The minimum level of detection of B-mtDNA varied with RNAse treatment of the extract, concentration (%) of BMBM and complexity of the feed. RNAse treatment of each sample decreased the overall false negative results 75%. RNAse treatment dramatically decreased false negative results 100% in samples containing 2%, 1% and 0.5% BMBM. At the 0.2% and 0.1% levels the false negative results decreased 50%.

Confirmation of the amplification of a 283 bp product validates the bovine specific primers and the use of real-time light cycler technology (FIG. 1). PCR products from cattle feeds spiked with 1% and 0.5% BMBM and the two positive BMBM controls display strong peaks at the same temperature, although with slightly lower cross-over points, (understandably, since the concentration of the ampligen is less in the extracts than in the positive controls). PCR products from cattle feed spiked with 0.01% and 0.001% BMBM did not amplify. Gel electrophoresis of the PCR products demonstrates the same result. A 300 bp DNA ladder band was comparable to the bands developed with PCR products from cattle feed spiked with the 0.1% and 0.05% BMBM, and with the two positive control BMBM products but missing with the negative control and PCR products from cattle feed spiked with 0.01% and 0.001% BMBM.

### Example 5

### The use of FRET Probe Technology in Real Time Fluorescent PCR to Detect and Differentiate Ruminant Species DNA

In order to detect and differentiate between bovine, sheep, and goat species DNA in a single PCR reaction, a set of FRET probes (SEQ ID NOS:13 and 14) and primers (SEQ ID NOS:11 and 12) were designed and used in a similar fashion as described by Roche for mutational analysis using the Lightcycler system (Roche Molecular Biochemicals).

The technique of mutational analysis using the Roche Lightcycler is based on the principal that during the heating of PCR products, sequence specific FRET probes will melt off at defined temperatures. The temperature at which the probes dissociate from the target DNA (usually defined as the Tm, the temperature at which 50% of the probe has dissociated from the target DNA) is directly related to both the sequence homologies between the probes and target sequence and the size of the probes. At 100% sequence homology between the probes and target sequence, the probes will remain annealed to the target sequence up to a maximum temperature. In the event of a single base mismatch between the probes and target sequence, the stability of the annealed probes will decrease, thus resulting in a lower temperature at which the probes will melt off of the target sequence. Roche describes this method for the screening of wild type and mutant DNA by comparing the differences in the resulting melting curves.

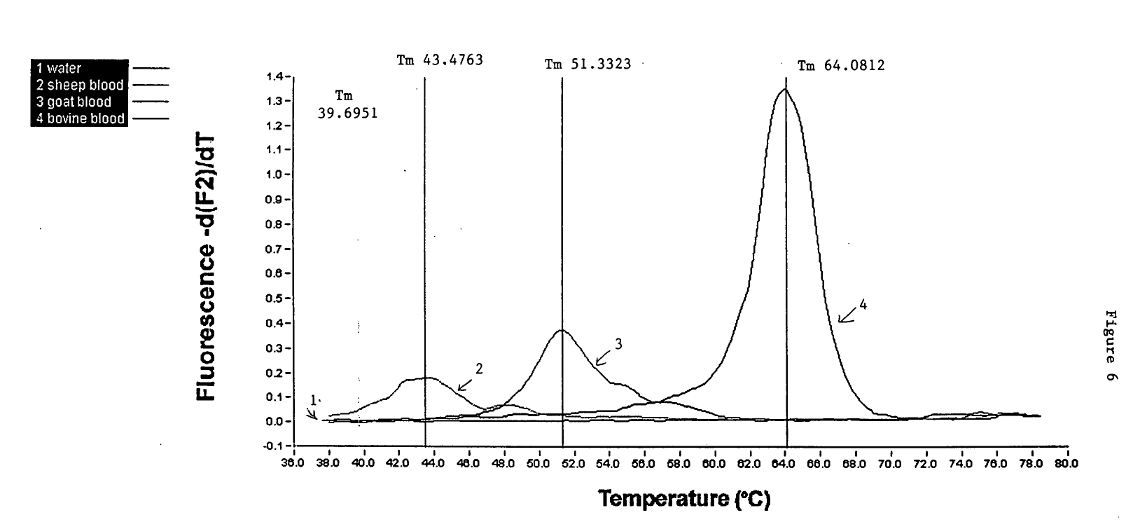

We used a modification of this approach to distinguish between the sequence differences of the DNA amplified with a single set of primers, thus allowing the identification of bovine, sheep, and goat DNA resulting from one PCR amplification. A single set of primers and probes were designed so that the DNA from all three species of ruminants would amplify, and the probes would bind to all three amplicons but with varying degrees of homology. The FRET probes bind to bovine target sequence with 100% homology, goat target sequence with 93% homology and sheep target sequence with 88% homology. The differences in homology result in three distinct melting curve temperatures (Tm), each corresponding to bovine, goat, or sheep species. The results are shown in FIG. 6.

The FRET probe technology can conveniently be used in conjunction with RNAse treatment as described herein to amplify and detect ruminant DNA.

### Example 6

### The Use of Nested PCR to Amplify Ruminant DNA

Nested PCR as described in, e.g., Aradaib et al, Vet. Sci. Animal Husbandry 37 (1-2): 13-23 (1998) and Aradaib et al., Vet. Sci. Animal Husbandry 37 (1-2): 144-150 (1998) can also be used to amplify target nucleic acid sequences. A first amplification step using an “outer” pair of primers (e.g., SEQ ID NOS:7 and 10) is used to amplify a highly conserved region of the target sequence (e.g., cytochrome b). A second amplification using an “inner” (i.e., “nested”) pair of primers (e.g., SEQ ID NOS:5 and 6 or 8 and 9) is used to amplify a portion of the target sequence (e.g., cytochrome b) that is contained within the first amplification product.

In particular, the SEQ ID NOS:7 and 10 can be used to amplify a 736 bp sequence from ruminant cytochrome b. SEQ ID NOS:8 and 9 can be used to amplify a 483 bp ruminant cytochrome b sequence within the 736 bp sequence amplified using SEQ ID NOS 7 and 10. SEQ ID NOS:5 and 6 can be used to amplify a 606 bp sheep cytochrome b sequence within the 736 bp sequence amplified using SEQ ID NOS:7 and 10.

The nested PCR can conveniently be used in conjunction with RNAse treatment described herein to amplify and detect ruminant DNA.

These studies addresses the “real life” conditions and problems encountered in the detection of banned components in animal feed or animal feed components. In particular, it confirms that different results are obtained with cattle feeds of varying complexities. These differences are attributable to inhibiting substances extracted simultaneously with the target DNA. Typical measures taken during extraction to decrease the amount of inhibitors may not be completely effective and therefore an internal control to detect the presence of any PCR inhibitor can be included in the reaction mixture. Identification and diminution or elimination of the substance causing inhibition can improve consistency and detection.

“Spiking” the feeds with rendered animal products represents incorporation of the most frequently used components added to cattle feed, again simulating field conditions.

When the presence of inhibiting substances is taken into consideration, the use of highly specific primers combined with fluorescent real time PCR technology offers the potential for the solution to detection and identification of minute amounts of banned products contained in various cattle feeds.

### Example 7

### Comparison of PCR-Based and Antibody-Based Detection of Bovine Byproduct Contamination of Cattle Feeds

We compared the polymerase chain reaction (PCR)-based method for detecting ruminant nucleic acid in samples (see, e.g., Sawyer et al., J. Foodborne Pathogens and Disease 1(2):105-113 (2004) and Example 3 above) with an antibody based method for detecting ruminant peptides in samples (i.e., Reveal® for Ruminant Detection (Neogen Corporation, Lansing Mich.). Comparison of the two different technologies using the same feeds “spiked, with banned additives of either Bovine Meat and Bone Meal (BMBM) or Bovine Dried Blood (BDB) demonstrated that consistent detection of smaller amounts of contamination was more. likely with a more sensitive quantitative PCR analysis

More particularly, we investigated the efficacy of both technologies in detecting the presence of bovine tissues in a variety of cattle feeds and compared results using five representative cattle feeds “spiked” with predetermined concentrations of either bovine meat and bone meal (BMBM) or bovine dried blood (BDB). Prior to PCR analysis, digestion of the samples and DNA extraction were performed using modifications of a commercial kit (Qiagen Plant Kit, Qiagen Inc, Valencia, Calif.). Detection and analysis were accomplished through fluorescent PCR using the Lightcycler (Roche Applied Sciences, Indianapolis, Ind.) and were performed on each concentration of BMBM and BDB. Quantitative PCR, using bovine specific mitochondrial primers and fluorescence resonance energy transfer (FRET) probes is described in detail in Example 5 above. The Reveal® kit was used according to manufacturer's instructions.

Five representative cattle feeds were included in this study. The ratio of concentrate to roughage for each feed is described as follows:
#1 Finishing Ration I: 80%: 20%, without molasses and bovine tallow;
#2 Finishing Ration II: 80%: 20% with molasses and bovine tallow;
#3 Starter Calf Ration: 40%: 60%;
#4 Grower Calf Ration: 60%: 40%; and
#5 Weaning Calf Ration: 70%: 30% (“Calf Maker” Alderman-Cave Milling and Grain Company of New Mexico, Roswell, N.Mex.) a granular commercial ration

The feeds were “spiked” with either commercially rendered bovine meat and bone meal (BMBM) or bovine dried blood (BDB) as directed by each protocol. “Unspiked” feeds were included as negative controls.

One set samples of the five cattle feeds was processed according to the manufacturer's instructions for the Reveal® Strip Test Kit. The feeds were spikes by adding the appropriate amount of BMBM or BDB directly to the extraction vessel containing 10 gm of the feed. The spiked samples were swirled, then boiled for 10 minutes. An aliquot of the liquid was transferred to a microcentrifuge tube; a strip test was inserted and allowed to develop for precisely 10 minutes.

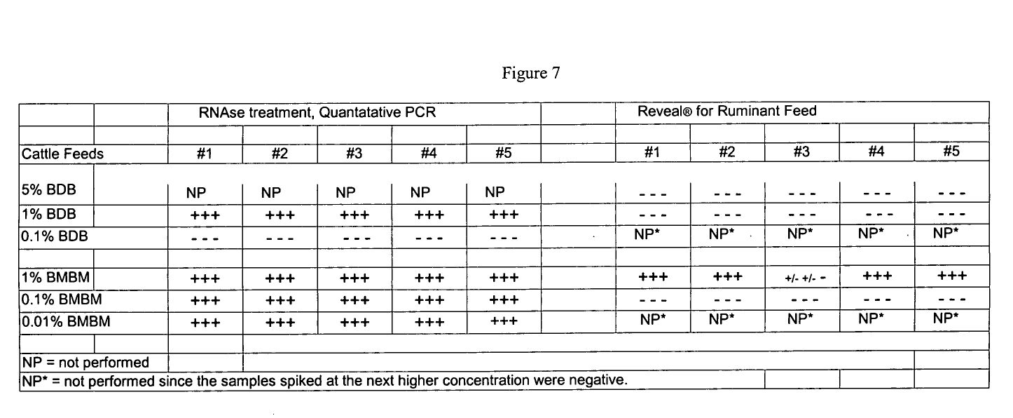

Another set of samples of the five cattle feeds was processed as follows: prior to PCR analysis, each feed sample was ground to a fine powder and spiked by adding the appropriate amount of BMBM or BDB. Digestion and extraction of DNA was accomplished using minor modifications of the Qiagen Plant Kit in which the protocol was adapted to accommodate a larger sample size (0.22 gm) and DNA and RNA free RNAse (Roche Applied Sciences, Indianapolis, Ind.) was added at a rate adjusted to the volume of the shredder column eluate. The extracted DNA was aliquoted and subjected to PCR analysis. The results are shown in FIG. 7.

As explained above, inhibitors, such as RNA, released from the feed during digestion have been implicated in causing false negative PCR results. Treatment of the extracted DNA with RNAse prior to PCR resulted in consistently more sensitive detection levels. (Sawyer et al., 2004, supra) The feeds containing the highest amounts of roughage appear to be most frequently associated with the presence of PCR inhibitors. The disparity in PCR results was consistently observed between the other feeds tested and feed #3, (60% roughage) and to a lesser extent with feed #4, (40% roughage). (Sawyer et al., 2004, supra) This inability to consistently achieve the lower detection levels of the other feeds was observed with both technologies.

The bovine mitochondrial DNA primers used for the PCR analysis detect only nucleated cells. Since only white blood cells are nucleated and red blood cells constitute the majority of the mass of dried blood, it is more difficult to detect ruminant DNA in feed spiked with BDB. Meat and bone meal products contain more nucleated cells. Thus, ruminant DNA was more likely to be detected in feed spiked with BMBM than in feed spiked with the same percentage of BDB. Similarly, the bovine tallow included in feeds #2 and #3 remained undetected in the unspiked negative control because of the paucity of nucleated cells and the low concentration ( 1.5% to 2.5% “fat”) present in the feed.

PCR technology consistently detected BMBM in all five feeds at the 1% and also at ten-fold less “spiking” (0.1%). BDB was similarly detected at the 1% level; however, all feed samples were negative when run at the 0.1% BDB “spiking” level.

The antibody-based Reveal® Strip Test detected BMBM at the 1% level in feeds #1,#2,#4 and #5, but results were inconclusive in feed #3. BMBM was not detected in any of the feeds at the 0.1% level. BDB was not detected in any of the five feeds at the 5% level (five-fold greater than the level detected with PCR). Since we found that the Reveal® Test produced negative results in feeds spiked with 5% BMBM, a concentration that is visually positive to the naked eye, we did not test samples spiked with 1% BMBM. Failure to consistently detect BMBM at a 1% level of “spiking” and BDB at a level of 5% “spiking” is a disadvantage in the Reveal® Test.

The results of the Reveal® Test at the minimal levels of detection are subjective and ambiguous. In all cases, a definite positive control line was apparent within 5 minutes, however most of the test samples required 10 minutes to develop a barely perceptible test sample line. In some samples, the intensity of the test sample line increased and became more apparent with an additional 10-15 minutes, but in all cases never attained the intensity of the positive test line. The later development of the sample line using the makes maintaining an accurate and permanent record using the stored test strips questionable.

Thus, the Reveal® Test can not be considered reliable for detection of ruminant contamination of samples at lower or unknown levels of contamination. Therefore, we conclude that PCR offers a more reliable, comprehensive tool.

### Example 8

### Development and Evaluation of a Real-Time Fluorescent PCR Assay for the Detection of Bovine Contaminants in Commercially Available Cattle Feeds

A real time fluorescent polymerase chain reaction assay for detecting prohibited ruminant materials such as bovine meat and bone meal (BMBM) in cattle feed using primers and FRET probes targeting the ruminant specific mitochondrial cytochrome b gene was developed and evaluated on two different types of cattle feed. Common problems involved with PCR based testing of cattle feed include the presence of high levels of PCR inhibitors and the need for certain pre-sample processing techniques in order to perform DNA extractions. We have developed a pre-sample processing technique for extracting DNA from cattle feed which does not require the feed sample to be ground to a fine powder and utilizes materials that are disposed of between samples, thus, reducing the potential of cross contamination. The DNA extraction method utilizes Whatman FTA® card technology, is adaptable to high sample throughput analysis and allows for room temperature storage with established archiving of samples of up to 14 years. The Whatman FTA® cards are subsequently treated with RNAse and undergo a Chelex-100 extraction (BioRad, Hercules, Calif.), thus removing potential PCR inhibitors and eluting the DNA from the FTA® card for downstream PCR analysis. The detection limit was evaluated over a period of 30 trials on calf starter mix and heifer starter ration feed samples spiked with known concentrations of bovine meat and bone meal (BMBM). The PCR detection assay detected 0.05% wt/wt BMBM contamination with 100% sensitivity, 100% specificity and 100% confidence. Concentrations of 0.005% and 0.001% wt/wt BMBM contamination were also detected in both feed types but with varying levels of confidence.

### Example 9

### Effect of RNAse Treatment on the PCR Cattle Feed Assay Using the FTA/Triple DNA Extraction Protocol

To determine the effect of RNase treatment on the diagnostic accuracy of a real time fluorescent PCR assay for detecting ruminant contaminants such as bovine meat and bone meal (BMBM) in cattle feed, we ran 30 samples plus and minus the RNAase treatment and performed statistical analysis.

Sample preparation: Thirty replicates were prepared in which commercially rendered BMBM was added at a concentration of 0.001% wt/wt to heifer starter ration. In order to obtain 0.001% BMBM, 0.003 g of BMBM was weighed on a Mettler AE 160 analytical balance then added to 300 g of the heifer starter ration. The 300 grams of spiked heifer starter ration was then weighed out into 10 g amounts for DNA extraction.

DNA extraction from cattle feed: 10 g feed samples were placed in a sterile 50 ml Falcon tube (Fisher Scientific, Pittsburgh, Pa.). A volume of 25 ml of cell lysis buffer made up of 5 M guanidinium isothiocyanate, 50 mM Tris-Cl, 25 mM EDTA, 0.5% Sarkosyl, 0.2M β-mercaptoethanol (Chakravorty and Tyagi, FEMS Microbiol. Lett. 205:113-117 (2001)) was added and the sample was vortexed. The sample was incubated at room temperature (RT) for 10 min. The sample was placed in a centrifuge and centrifuged at 17,000 ×g for 1 minute to recover the cell lysis buffer from the highly absorptive cattle feed. A volume of 65 μl of the cell lysis buffer was removed using a wide bore pipet tip and spotted onto a Whatman FTA® card (Whatman, Clifton, N.J., Cat #WB 12 0206) and dried at RT for 1 hr. A 2 mm Whatman punch was used to obtain two separate 2 mm disks containing the sample. Each of the thirty 2 mm disks were placed in a 1.5 ml sterile tube and labeled 1-30 RNase treated and 1-30 non-RNase treated.

RNase treatment: 100 μl of RNase (DNA-free RNase; Roche Applied Science, Indianapolis, Ind., Cat # 1119915) at a concentration of 0.05 μg/μl was added to each of the 1.5 ml sterile tubes labeled 1-30 RNase treated. The tubes were placed in a heating block and allowed to incubate at 37° C. for 1 hr. After incubation the 100 μl of RNase was removed from the tube and discarded. 200 μl of Instagene (BioRad, Hercules, Calif., and Cat # 732-0630) was added and the samples were placed in a heating block at 56° C. for 30 min. The samples were removed from the heating block and vortexed for 10 sec. The samples were then placed in a 100° C. heating block for 8 min. The samples were then vortexed and centrifuged at 12,000 ×g for 3 min. The supernatant was removed and placed in a new sterile 1.5 ml tube for PCR analysis.

Non-RNase treatment: 200 μL of FTA purification reagent (Cat# WB12 0204) was added to each of the 1.5 ml sterile tubes labeled 1-30 Non-RNase treated. The tubes were then incubated for 5 min. at RT. The FTA purification reagent was then discarded and the process was repeated for a total of two washes. 200 μl of TE-1 Buffer (10 Tris-HCl, 0.1 mM EDTA, pH 8.0) was then added and the tube was incubated at RT for 5 minutes. The TE-1 buffer was discarded and the process was repeated for a total of two washes. 200 μl of Instagene (BioRad, Hercules, Calif., Cat# 732-0630) was added and the samples were placed in a heating block at 56° C. for 30 min. The samples were removed from the heating block and vortexed for 10 sec. The samples were then placed in a 100° C. heating block for 8 min. The samples were then vortexed and centrifuged at 12,000 ×g for 3 min. The supernatant was removed and placed in a new sterile 1.5 ml tube for PCR analysis.

Standard FRET PCR protocol: PCR reactions were run at a final concentration of 0.5 μM forward primer, 0.5 μM reverse primer, 0.2uM fluorescein labeled probe, 0.4 μM LC-Red 640 labeled probe, 3 mM MgCl₂, and 1× LightCycler Fast Start DNA master Hybridization probes mix. The DNA samples were added in 5 μl volumes to the reaction mixture for a total of 20 μl in each reaction. All sixty PCR reactions were run simultaneously using the Corbett Roto-Gene 3000. The conditions for cycling were 95° C. for 10 min. (denaturation and Taq. polymerase activation) followed by an amplification program of 50 cycles at 95° C. for 0 Sec., 55° C. for 12 sec., and 72° C. for 14 sec. LC-Red 640 was monitored at the end of each 55° C. step. The amplification program was then followed with 1 melting cycle of 95° C. for 30 sec., 38° C. for 30 sec. and 80° C. for 0 sec with a transition rate of 0.1° C./sec.

The determination of a PCR positive result, was made based on the presence of an amplification curve and a melting curve with a melting temperature (Tm) between 62° C. and 63° C. A Tm between 62° C. and 63° C. represents hybridization with 100% homology between the probes and bovine mtDNA sequence.

The results of our assay to detect ruminant DNA derived from BMBM at a concentration of 0.001% wt/wt in heifer starter ration with and without the use of RNase were compared by using McNemar's test for correlation proportions (Remington and Schork: Statistics with Applications to the Biological & Health Sciences, 1970). At the 90% confidence level there was a significant effect (0.05<p<0.1) between the use of RNase treatment and the proportion of PCR positive results when compared to not treating the samples with RNase (Table 6). 26.7% of the samples treated with RNase were found to be PCR positive compared to 6.7% PCR positive samples without RNase treatment (Table 7).

TABLE 6
PCR results of thirty samples of heifer starter ration
spiked with BMBM at
0.001% wt/wt treated with RNase and not treated with RNase.

| | RNase Treatment | | |
|---|---|---|---|
| No RNase treatment | Positive | Negative | Total |
| Positive | 0 | 2 | 2 |
| Negative | 8 | 20 | 28 |
| Total | 8 | 22 | 30 |

0.05 < p < 0.1

TABLE 7
Individual sample PCR results of heifer starter
ration spiked with BMBM
at 0.001% wt/wt treated with RNase and not treated with RNase.
Heifer starter ration: ground and spiked at 0.001% BMBM

| Sample # | PCR results w/out RNAse | PCR Results with Rnase |
|---|---|---|
| 1 | Neg. | Neg. |
| 2 | Neg. | Positive |
| 3 | Neg. | Positive |
| 4 | Neg. | Positive |
| 5 | Neg. | Positive |
| 6 | Neg. | Neg. |
| 7 | Neg. | Neg. |
| 8 | Neg. | Neg. |
| 9 | Neg. | Neg. |
| 10 | Neg. | Neg. |
| 11 | Neg. | Neg. |
| 12 | Neg. | Positive |
| 13 | Neg. | Neg. |
| 14 | Neg. | Neg. |
| 15 | Neg. | Neg. |
| 16 | Neg. | Neg. |
| 17 | Positive | Neg. |
| 18 | Positive | Neg. |
| 19 | Neg. | Neg. |
| 20 | Neg. | Positive |
| 21 | Neg. | Neg. |
| 22 | Neg. | Neg. |
| 23 | Neg. | Neg. |
| 24 | Neg. | Neg. |
| 25 | Neg. | Neg. |
| 26 | Neg. | Positive |
| 27 | Neg. | Neg. |
| 28 | Neg. | Neg. |
| 29 | Neg. | Neg. |
| 30 | Neg. | Positive |

### Example 10

Detection of Ruminant DNA in a Vaccine Sample Using RNAse Treatment and the FTA/Triple DNA Extraction Protocol

To evaluate the detection limits of the current bovine PCR detection assay when applied to the E.coli Bacterin J5 strain vaccine (Upjohn) and to evaluate the effects of the E. coli Bacterin J5 strain vaccine (Upjohn) on PCR reaction efficiency using Real-Time Fluorescent Quantitative PCR targeting the bovine mitochondrial cytochrome b gene, the following experiments were conducted.

Bovine DNA Standard: A bovine DNA standard was prepared by extracting DNA from bovine meat and bone meal (BMBM) and quantitated with a spectrophotometer.

DNA extraction from E. coli Bacterin J5 strain vaccine (Upjohn): 65 μL of the E. coli J5 vaccine was applied to an FTA card and the DNA extraction protocol described in Example 9 above was followed. The DNA extract was then quantitated with a spectrophotometer. The concentration and the 260/280 ratio was used in order to verify that DNA was isolated from the E. coli J5 vaccine.

Preparation of Serial Dilutions: A series of four ten fold serial dilutions were prepared in which 10 μL of the bovine DNA standard was diluted into 90 μL of the E. coli J5 DNA extract.

Real-Time PCR: PCR was run on the four serial dilutions including the non-diluted bovine DNA standard. The experiment was repeated for a total of three times.

The concentration of the bovine DNA standard was determined to be 50 ng/μl with a 260/280 ratio of 2.00 and the concentration of the DNA extracted from the E. coli J5 vaccine was determined to be 6.57 ng/μl with a 260/280 ratio of 1.77.

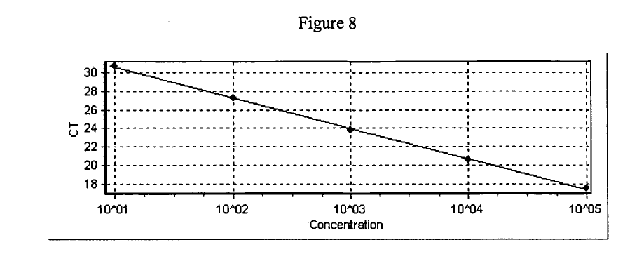

For the Real-Time PCR, the threshold values in relation to the log of the DNA concentrations were used in order to construct a graph (FIG. 8.) The efficiency of the PCR reaction was calculated based on the slope of the line. The PCR assay was able to detect 5 pg/μL of bovine DNA with an average PCR efficiency of 99% over three trials (Table 8.).

TABLE 8
PCR reaction efficiencies of bovine DNA standard serially diluted into
DNA extract from E. coli Bacterin J5 strain vaccine (Upjohn).

| Experiment # | PCR reaction efficiency |
|---|---|
| 1 | 98% |
| 2 | 100% |
| 3 | 99% |

It is understood that the examples and embodiments described herein are for illustrative purposes only and that various modifications and changes in light thereof will be suggested to persons skilled in the art and are to be included within the purview of this application and are considered to be within the scope of the appended claims. All publications, patents, and patent applications cited herein are hereby incorporated by referenced in their entirety for all purposes.

1. A method of amplifying ruminant DNA in a sample, said method comprising:
contacting nucleic acid from said sample with an RNase, thereby generating RNase-treated nucleic acid; and
amplifying said RNAse-treated nucleic acid using a first ruminant-specific primer and a second-ruminant-specific primer, thereby amplifying ruminant DNA present in said sample and producing an amplified ruminant DNA.

2. The method of claim 1, wherein said nucleic acid is isolated from said animal feed prior to contacting said nucleic acid with an RNase.

3. The method of claim 1, wherein said ruminant DNA is a member selected from the group consisting of: cattle DNA, sheep DNA, goat DNA, and combinations thereof.

4. The method of claim 1, wherein said RNase is a member selected from the group consisting of: RNase A, RNase B, RNase D, RNase E, RNase H, RNase I, RNase P, RNase S, RNase T, RNase V, and combinations thereof.

5. The method of claim 1, wherein said RNase-treated nucleic acid is generated by contacting said isolated nucleic acid with said RNase at about 30° C. to about 40° C. for about 15 minutes to about 120 minutes.

6. The method of claim 1, wherein said RNase-treated nucleic acid is generated by contacting said isolated nucleic acid with said RNase at about 37° C. for about 60 minutes.

7. The method of claim 1, wherein said ruminant DNA comprises a mitochondrial DNA sequence.

8. The method of claim 7, wherein said mitochondrial DNA sequence encodes a member selected from the group consisting of: cytochrome c, cytochrome b, 12S RNA, ATPase subunit 8, ATPase subunit 6, ATP synthetase, subunit 8, and subsequences and combinations thereof.

9. The method of claim 8, wherein said mitochondrial DNA sequence encodes cytochrome b or a subsequence thereof.

10. The method of claim 1, wherein said first ruminant-specific primer and said second ruminant-specific primer are selected from the group consisting of: SEQ ID NOS:1 and 2, SEQ ID NOS:3 and 4, and SEQ ID NOS:11 and 12.

11. The method of claim 1, further comprising detecting said amplified ruminant DNA.

12. The method of claim 11, wherein detecting said amplified ruminant DNA comprises detecting a fluorescent signal.

13. The method of claim 11, wherein detecting said amplified ruminant DNA comprises contacting said amplified ruminant DNA with an oligonucleotide probe.

14. The method of claim 13, wherein said ruminant DNA is amplified using a first ruminant-specific primer and a second-ruminant-specific primer comprising the sequences set forth in SEQ ID NOS:11 and 12 and detecting said amplified ruminant DNA comprises contacting the amplified ruminant DNA with oligonucleotide probes comprising the sequences set forth in SEQ ID NOS:13 and 14.

15. The method of claim 1, further comprising amplifying said amplified ruminant DNA with a third ruminant-specific primer and a fourth-ruminant-specific primer, thereby producing a second amplified ruminant DNA.

16. The method of claim 15, further comprising detecting said second amplified ruminant DNA.

17. The method of claim 1, wherein said sample is a member selected from the group consisting of: an animal feed, an animal feed component, a cosmetic, a nutraceutical, a vaccine, a colloidal infusion fluid, or combinations thereof.

18. The method of claim 1, wherein said sample is an animal feed.

19. The method of claim 18, wherein said animal feed is cattle feed.

20. The method of claim 19, wherein said cattle feed comprises about 0.5% to about 30% bovine tallow.

21. The method of claim 19, wherein said cattle feed comprises about 1% bovine tallow.

22. The method of claim 1, wherein said sample is an animal feed component.

23. The method of claim 22, wherein said animal feed component is beef tallow.

24. A kit for amplifying ruminant DNA, said kit comprising:
a first pair of ruminant-specific primers;
an RNAse; and
instructions for use.

25. The kit of claim 24, wherein said RNase is a member selected from the group consisting of: RNase A, RNase B, RNase D, RNase E, RNase H, RNase I, RNase P, RNase S, RNase T, RNase V, and combinations thereof.

26. The kit of claim 24, wherein said first pair of ruminant-specific primers is selected from the group consisting of the sequences set forth in SEQ ID NOS:1 and 2; SEQ ID NOS:3 and 4; and SEQ ID NOS:11 and 12.

27. The kit of claim 24, further comprising a second pair of ruminant-specific primers.

28. The kit of claim 27, wherein said first pair of ruminant-specific primers is selected from the group consisting of the sequences set forth in SEQ ID NOS:1 and 2 and SEQ ID NOS:3 and 4, and said second pair of ruminant-specific primers is selected from the group consisting of the sequences set forth in SEQ ID NOS:1 and 2; and SEQ ID NOS:3 and 4.

29. The kit of claim 24, further comprising an oligonucleotide probe for detecting an amplified target sequence.

30. The kit of claim 29, wherein the oligonucleotide probe comprises a sequence selected from the group consisting of: SEQ ID NO: 13 and 14.

31. An isolated nucleic acid comprising the nucleic acid sequence set forth in SEQ ID NOS:1, 2, 3, 4, 11, 12, 13, or 14.


SEQUENCE LISTING (14 sequences)

SEQ ID NO: 1 - DNA, 19 bp, Artificial Sequence
  Description of Artificial SequenceCSL1 B cattle specific primer 1, ruminant-specific primer
gaatttcggttccctcctg

SEQ ID NO: 2 - DNA, 19 bp, Artificial Sequence
  Description of Artificial SequenceCSR2 B cattle specific primer 2, ruminant-specific primer
ggctattactgtgagcaga

SEQ ID NO: 3 - DNA, 20 bp, Artificial Sequence
  Description of Artificial SequenceGSL1 B goat-specific primer 1, ruminant-specific primer
tcatacatatcggacgacgt

SEQ ID NO: 4 - DNA, 20 bp, Artificial Sequence
  Description of Artificial SequenceGSR2 B goat-specific primer 2, ruminant-specific primer
caagaattagtagcatggcg

SEQ ID NO: 5 - DNA, 25 bp, Artificial Sequence
  Description of Artificial Sequencesheep specific primer 1, nested PCR cytochrome b second amplification “inner” primer
catttgcttaattttacagattcta

SEQ ID NO: 6 - DNA, 28 bp, Artificial Sequence
  Description of Artificial Sequencesheep specific primer 2, nested PCR cytochrome b second amplification “inner” primer
catgaggatgaggattagtaggatagca

SEQ ID NO: 7 - DNA, 29 bp, Artificial Sequence
  Description of Artificial Sequenceruminant specific primer 1, nested PCR cytochrome b first amplification step “outer” primer
tcgaaagtcccacccactaataaaaattg

SEQ ID NO: 8 - DNA, 22 bp, Artificial Sequence
  Description of Artificial Sequenceruminant specific primer 2, nested PCR cytochrome b second amplification “inner” primer
ttgaagctccgtttgcgtgtat

SEQ ID NO: 9 - DNA, 25 bp, Artificial Sequence
  Description of Artificial Sequenceruminant specific primer 3, nested PCR cytochrome b second amplification “inner” primer
tcagattcattcgactaaatttgtg

SEQ ID NO: 10 - DNA, 23 bp, Artificial Sequence
  Description of Artificial Sequenceruminant specific primer 4, nested PCR cytochrome b first amplification step “outer” primer
ggaggttgggcgcaaatagtact

SEQ ID NO: 11 - DNA, 16 bp, Artificial Sequence
  Description of Artificial Sequencesecond generation ruminant primer 1, PCR FRET primer, ruminant-specific primer
tacacgcaaacggagc

SEQ ID NO: 12 - DNA, 16 bp, Artificial Sequence
  Description of Artificial Sequencesecond generation ruminant primer 2, PCR FRET primer, ruminant-specific primer
gagcctgtttcgtgga

SEQ ID NO: 13 - DNA, 23 bp, Artificial Sequence
  Description of Artificial SequencePCR FRET probe 1 (fluorescein)
caatcccatacatcggcacaaan

SEQ ID NO: 14 - DNA, 21 bp, Artificial Sequence
  Description of Artificial SequencePCR FRET probe 2 (Red 640)
agtcgaatgaatctgaggcgn